(19) United States
(12) Patent Application Publication
(10) Pub. No.: US 2023/0407314 A1
(43) Pub. Date: Dec. 21, 2023

(54) ANAEROBIC CO-PRODUCTION OF ESSENTIAL AMINO ACIDS, ALCOHOLS AND LIPIDS FROM MOLASSES, HYDROLYSED STARCH AND LIGNOCELLULOSE

(71) Applicant: Ambareesh Govind Phadnavis, Evershine City, Vasai (east) (IN)

(72) Inventor: Ambareesh Govind Phadnavis, Evershine City, Vasai (east) (IN)

(21) Appl. No.: 18/035,068

(22) PCT Filed: Nov. 2, 2021
(86) PCT No.: PCT/IN2021/051045

§ 371 Date: May 2, 2023

(51) Int. Cl. C12N 15/52; C12P 7/6409; C12P 7/6445; C12P 13/08; C12P 7/06; C12N 9/88; C12N 9/10; C12N 9/04; C12N 9/12; C12N 9/16; C12N 9/00

(52) U.S. Cl. CPC C12N 15/52; C12P 7/6409; C12P 7/6445; C12P 13/08; C12P 7/06; C12N 9/88; C12Y 401/01032; C12Y 402/01036; C12N 9/1096; C12Y 206/01001; C12N 9/1025; C12Y 203/03001; C12N 9/0006; C12Y 101/01087; C12Y 101/01008; C12Y 101/01042; C12Y 101/09001; C12Y 401/02048; C12N 9/1029; C12Y 203/01054; C12Y 206/01042; C12N 9/1205; C12Y 207/01086; C12N 9/16; C12Y 301/02014; C12N 9/93; C12Y 604/01002

(57) ABSTRACT

The invention provides a genetically modified eukaryotic microorganism for anaerobic production of essential amino acids and optionally the co-production of one or more co-products. The microorganism is genetically modified to redirect carbon flow from PEP via oxaloacetate and asparatate semialdehyde, towards the synthesis of increased amounts of essential amino acids. The microorganism may be genetically modified to produce increased amounts of one or more co-product by enhancing carbon flow from PEP via pyruvate, acetyl CoA and malonyl CoA to produce alcohols and lipids, such as triglycerides, fatty esters, fatty alcohols, fatty aldehydes, fatty amides. The invention provides a method for anaerobic production of essential amino acids using the genetically modified eukaryotic microorganism and optionally co-production of said one or more co-products. The genetically modified eukaryotic microorganism may be used for the anaerobic production of essential amino acids and optionally the co-production of said one or more co-products.

### BACKGROUND

### Technical Field

The invention provides a genetically modified eukaryotic microorganism for anaerobic production of essential amino acids and co-production of one or more co-products. The microorganism of the invention is genetically modified to redirect carbon flow from PEP via oxaloacetate and asparatate semialdehyde, towards the synthesis of increased amounts of essential amino acids. The microorganism may be further genetically modified to produce increased amounts of one or more co-product by enhancing carbon flow from PEP via pyruvate, acetyl CoA and malonyl CoA to produce alcohols and lipids, such as triglycerides, fatty esters, fatty alcohols, fatty aldehydes, fatty amides. The invention further provides a method for anaerobic production of essential amino acids using the genetically modified eukaryotic microorganism and co-production of said one or more co-products. Furthermore, the genetically modified eukaryotic microorganism may be used for the anaerobic production of essential amino acids and co-production of said one or more co-products.

### Description of the Related Art

The ongoing “Food or Fuel” crisis, Depleting petroleum reserves, increasing population and the stresses it puts to meet the necessities (nutritional and energy) on the existing technological and logistical infrastructure, recurrent energy crises, increasing demand, and climate change have provided significant impetus in the search for sustainable technologies to replace petroleum as a source of fuels and chemical feedstocks. Ethanol and Oils have been primary contenders for meeting sustainable energy demands. In particular, cellulosic biomass is a preferred source of generating ethanol and lipids and other derivatives for use as fuel and chemical feedstocks, which are compatible with existing petroleum refining and distribution and can substitute for diesel, gasoline, jet fuel, and other derivatives of crude oil.

Similarly, the term “food OR fuel” inherently means that it is a zero-sum game. The demand for protein rich food and feed (for humans and domesticated animals, fish, poultry etc) is high. This is highlighted by the fact that mammals and birds cannot synthesize the 9-essential amino-acids and they have to be acquired by the animals from its regular diet. The shortfall of these 9 amino-acids is driving up the prices of high-quality protein rich feed.

Currently, commercial and academic efforts are focused on bio-based petroleum replacement fuels made from microorganisms such as microalgae and that require aerobic microbial production. Yeast based platforms are already operational to produce ethanol which is blended into petrol for bringing down carbon emissions. Algae bio-petroleum can appear as a very attractive option because fuel production occurs directly from sunlight and CO2. However, algal volumetric productivities are 100-fold lower than fermentative processes, requiring significantly higher biorefinery capital expenditures. In addition, lower capital algal options, such as open pond culturing, have many technical hurdles to clear before commercial deployment despite decades of research into the issue.

Unlike traditional ethanol fermentations, aerobic biofuel synthesis routes feature product formation which is uncoupled from ATP generation and cell growth. Uncoupling of product formation from cell growth simplifies metabolic engineering and has allowed for rapid development of first-generation biocatalysts. However, there is a price to be paid for aerobic production when the technology is scaled up to meet industrial needs. First, there are significant costs associated with scaling-up aerobic fermentations, such as, those due to the need for aeration and heat removal. In practice, these constraints limit the size of aerobic fermentors, with those used in anaerobic fuel ethanol production being an order of magnitude larger. Second, although maximum theoretical product yields from an aerobic process are only slightly lower than an anaerobic process, in practice it is extraordinarily difficult to approach this maximum since there is no biological incentive for microbes to reach high product yields.

An anaerobic, aeration/oxygen-free fermentation not only creates higher product yields, but also removes many significant scale-up problems associated with aerobic fermentation. Hydrocarbon fuel production also has process benefits compared to ethanol fuel production, such as a lower product recovery cost and a lower product toxicity to fermenting organisms. The latter could result in smaller fermentation volumes needed to reach equivalent productivities. An anaerobic bioprocess however requires a higher degree of metabolic pathway integration to couple product formation with ATP generation, NAD(P)H regeneration, and cell growth. Process economics plays a vital role in this scenario. Currently a bioethanol

producer is already selling its sludge as animal feed, oil as furnace oil and ethanol as fuel. However, by improving the quality (rich in essential amino acids, oil with smaller chain-length) and quantity of these by-products, would provide an additional revenue stream for the traditional 1G and 2G ethanol manufacturer.

The present invention addresses the need for providing a microbial cell that is engineered to produce added-value by-products during anaerobic fermentation as an additional revenue stream

### SUMMARY

According to a first embodiment, the invention provides a genetically modified eukaryotic microorganism, wherein the genome of said microorganism is modified relative to a parent non-modified microorganism to: express a heterologous phosphoenolpyruvate carboxykinase (Enzyme commission Number 4.1.1.32); increase expression of aspartate aminotransferase (Enzyme Commission Number 2.6.1.1); increase expression of homoaconitase

(Enzyme Commission number 4.2.1.36) activity; increase expression of homocitrate synthase activity (Enzyme commission number: 2.3.3.1); increase expression of homoisocitrate dehydrogenase (Enzyme commission number: 1.1.1.87). The genetically modified eukaryotic microorganism produces increased levels of essential amino acids as compared to said non-genetically modified parent microorganism when cultured under comparable anaerobic fermentation conditions.

According to a second embodiment, the invention provides a genetically modified eukaryotic microorganism, wherein the genome of said microorganism is further modified relative to said parent non-modified microorganism to: a. express heterologous bacterial genes encoding: i. holo-[acyl-carrier-protein] synthase (Enzyme Commission Number 2.7.8.7) ii. acyl carrier protein, 3-oxoacyl-[acyl-carrier-protein] synthase 1 (Enzyme Commission Number 2.3.1.41), iii. malonyl CoA-acyl carrier protein transacylase (Enzyme Commission Number 2.3.1.39), iv. 3-oxoacyl-[acyl-carrier-protein] reductase (Enzyme Commission Number 1.1.1.100), v. 3-oxoacyl-[acyl-carrier-protein] synthase 3 (Enzyme Commission Number 2.3.1.41), vi. enoyl-acyl carrier protein reductas (Enzyme Commission Number 1.3.1.39), and vii. 3-hydroxyacyl-[acyl-carrier-protein] dehydratase (Enzyme Commission Number 4.2.1.59); b. silence expression of native fatty Acid synthase complex (Enzyme Commission Number 2.3.1.86/4.2.1.59); c. silence or reduce expression of native fatty acyl-CoA synthetase activity (Enzyme Commission Number 6.2.1.3); and d. increase expression of acetyl CoA carboxylase (Enzyme Commission Number 6.4.1.2); wherein the genetically modified eukaryotic microorganism co-produces increased levels of lipids as compared to said non-genetically modified parent microorganism when cultured under comparable anaerobic fermentation conditions.

According to a third embodiment, the invention provides “Dried Distillers Grains with Solubles” (DDGS) and/or high protein concentrate comprising the genetically modified eukaryotic microorganism of the invention.

According to a fourth embodiment, the invention provides a method for production of essential amino acids comprising: providing a genetically modified eukaryotic microorganism of the invention; culturing said microorganism in a nutrient medium under anaerobic conditions; and recovering one or more fractions of the culture obtained in step (b) enriched in essential amino acids.

According to a fifth embodiment, the invention provides a method, wherein said method is for co-production of lipids comprising: providing a genetically modified eukaryotic microorganism of the invention; culturing said microorganism in a nutrient medium under anaerobic conditions; and recovering one or more fractions of the culture obtained in step (b) enriched in essential amino acids and lipids.

According to a sixth embodiment, the invention provides a method according to the invention, wherein said nutrient medium comprises a terminal electron acceptor selected from among a nitrate, a nitrite, a fumarate, a chlorate, a perchlorate or a hypochlorous acid ion. According to a seventh embodiment, the invention provides Use of a genetically modified eukaryotic microorganism of the invention for co-production of essential amino acids and one of more co-product.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein, and the embodiments herein include all such modifications.

### BRIEF DESCRIPTION OF THE DRAWINGS

The embodiments herein will be better understood from the following detailed description with reference to the drawings, in which:

FIG. 1 is an overview of the added-value products produced by the genetically-modified microorganism and anaerobic fermentation process according to the invention;

FIG. 2 is an Essential amino acid biosynthesis pathway upregulation scheme. A bold arrows represent upregulation of a metabolic pathway in the genetically modified microorganism of the invention. A cross represents genetic downregulation of a catabolic pathway for production and secretion of a metabolic product according to some embodiments herein;

FIG. 3 is an anaerobic lipid co-production pathway upregulation scheme. A bold arrow indicates a gene that is overexpressed (a native or heterologously expressed gene). A crossed pathway indicates downregulation of expression of a gene either by gene-deletion or placing the gene under the control of a weak promotor; and

FIG. 4 is an Anaerobic pathways for the co-production of alcohol with essential amino-acids and lipids in the genetically modified microorganism of the invention.

FIG. 5 is a graph showing the growth curve of a parent wild-type yeast strain, measured as dry cell weight (DCW), during anaerobic fermentation growth on Yeast Nitrogen Base (YNB) defined media, further showing the profile of dextrose consumption and lysine, ethanol, and extracellular lipid secreted by the yeast strain. The unmodified strain was grown on (YNB) medium on one liter working volume in erlenmeyer conical flasks of 5 liter volume in triplicates. Ergo

FIG. 6 is a graph showing the growth curve of a genetically modified yeast strain (Final strain) of the invention, measured as dry cell weight (DCW), during anaerobic fermentation growth on a YNB defined media, further showing the profile of dextrose consumption and lysine, ethanol, and extracellular lipid secreted by the yeast strain. Final Strain Genotype: Δfaa2, Δfaa?, Δfaa1, Δfaa4+PACC1::PTEF1+PFLA+PFLB+Bacterial FAS Cassette+Palm Thioesterase::Yeast Thioesterase+PADH::PNADHKinase+Bacterially Krebs Cycle genes+ΔGPDH1+ΔADH1+ΔADH5+ΔGLY1+PGPD1::PAAT1+bacterial PCK1+PLY S20: :PCYCl+PADH2::PLYS4+Lys20+PLYS12::PZEV1+PLY S21::PPGK1+Cgl024 8.

FIG. 7 is a graph showing the growth curve of a genetically modified yeast strain (ARO8 strain) of the invention, measured as dry cell weight (DCW), during anaerobic fermentation growth on a YNB defined media, further showing the profile of dextrose consumption and lysine, ethanol, and extracellular lipid secreted by the yeast strain. ARO8 Strain Genotype:
Δfaa2+Δfaa3+Δfaa1+PACC1::PTEF1+ADH5::PFLA_AND_PFLB+Yeast_Thioesterase::Cuphea wrightii Thioesterase+GPD2::Bacterial FAS Cassette+PNADKinase::PADH1+ADH1::Bacterial Krebs Cycle Genes Cassette+ΔGPD2+ΔADH1+ΔADH5+ΔGLY1+PAAT1::PGPD1+YeastPCK1::bacterial PCK1+PLYS20::PCYCl+PLYS4::PADHl+PLYS12::PZEV1-2+FAA1:: cg10248+FAA3::PP3:ARO8

FIG. 8 is a Histogram showing a comparison of the lysine yield of a parent wild-type yeast strain and genetically modified yeast strains (ARO8 and Final strain) derived from the parent strain, expressed as A: grams lysine per gram of yeast dry cell weight and B: grams lysine per gram glucose consumed.

FIG. 9 is a Histogram showing a comparison of the lipid yield of a parent wild-type yeast strain and genetically modified yeast strains (ARO8 and Final strain) derived from the parent strain, expressed as grams total lipids [comprising free fatty acids and triglycerides] per gram of glucose consumed.

FIG. 10 is a Histogram showing a comparison of the ethanol yield of a parent wild-type yeast strain and genetically modified yeast strains (ARO8 and Final strain) derived from the parent strain, expressed as grams total ethanol per gram of glucose consumed.

FIG. 11 is a Histogram showing comparison of final fatty acid chain-length profile of lipids (Free fatty acids+Triglycerides) produced by parent wild-type yeast strain and genetically modifiec yeast strains (ARO8 and Final strain) derived from the parent strain, expressed as percentage of total lipids (free fatty acids and triglycerides) produced by the respective strains.

### DEFINITIONS

The indefinite articles “a” and “an” preceding an element or component of the invention are intended to include plurals of the element or component, e.g., one or at least one of the element or components, unless the context is such that only the singular form is intended.

The term “heterologous” when used in reference to a polynucleotide, a gene, a polypeptide, or an enzyme refers to a polynucleotide, gene, polypeptide, or an enzyme not normally found in the host organism. “Heterologous” also includes a native coding region, or portion thereof, that is reintroduced into the source organism in a form that is different from the corresponding native gene, e.g., not in its natural location in the organism's genome. The heterologous polynucleotide or gene may be introduced into the host organism by, e.g., gene transfer. A heterologous gene may include a native coding region that is a portion of a chimeric gene including non-native regulatory regions that is reintroduced into the native host. Foreign genes can comprise native genes inserted into a non-native organism, or chimeric genes.

The term “heterologous polynucleotide” is intended to include a polynucleotide that encodes one or more polypeptides or portions or fragments of polypeptides. A heterologous polynucleotide may be derived from any source, e.g., eukaryotes, prokaryotes, viruses, or synthetic polynucleotide fragments.

The terms “promoter” or “surrogate promoter” is intended to include a polynucleotide that can transcriptionally control a gene-of-interest that it does not transcriptionally control in nature. In certain embodiments, the transcriptional control of a surrogate promoter results in an increase in expression of the gene-of-interest. In certain embodiments, a surrogate promoter is placed 5′ to the gene-of-interest. A surrogate promoter may be used to replace the natural promoter, or may be used in addition to the natural promoter. A surrogate promoter may be endogenous with regard to the host cell in which it is used, or it may be a heterologous polynucleotide sequence introduced into the host cell, e.g., exogenous with regard to the host cell in which it is used.

The term's “gene(s)” or “polynucleotide” or “polynucleotide sequence(s)” are intended to include nucleic acid molecules, e.g., polynucleotides which include an open reading frame encoding a polypeptide, and can further include non-coding regulatory sequences, and introns. In addition, the terms are intended to include one or more genes that map to a functional locus. In addition, the terms are intended to include a specific gene for a selected purpose. The gene may be endogenous to the host cell or may be recombinantly introduced into the host cell, e.g., as a plasmid maintained episomally or a plasmid (or fragment thereof) that is stably integrated into the genome. In addition to the plasmid form, a gene may, for example, be in the form of linear DNA. The term gene is also intended to cover all copies of a particular gene, e.g., all of the DNA sequences in a cell encoding a particular gene product.

The term “transcriptional control” is intended to include the ability to modulate gene expression at the level of transcription. In certain embodiments, transcription, and thus gene expression, is modulated by replacing or adding a surrogate promoter near the end of the coding region of a gene-of-interest, thereby resulting in altered gene expression. In certain embodiments, the transcriptional control of one or more genes is engineered to result in the optimal expression of such genes, e.g., in a desired ratio. The term also includes inducible transcriptional control as recognized in the art.

The term “expression” is intended to include the expression of a gene at least at the level of mRNA production.

The term “expression product” is intended to include the resultant product, e.g., a polypeptide, of an expressed gene.

The term “polypeptide” is intended to encompass a singular “polypeptide,” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the amino acids. Thus, peptides, dipeptides, tripeptides, oligopeptides, “protein,” “amino acid chain,” “enzyme,” or any other term used to refer to a chain or chains of two or more amino acids, are included within the definition of “polypeptide,” and the term “polypeptide” may be used instead of, or interchangeably with, any of these terms. A polypeptide may be derived from a natural biological source or produced by recombinant technology. It may be generated in any manner, including by chemical synthesis.

The term “increased expression” is intended to include an alteration in gene expression at least at the level of increased mRNA production and, preferably, at the level of polypeptide expression. The term “increased production” is intended to include an increase in the amount of a polypeptide expressed, in the level of the enzymatic activity of the polypeptide, or a combination thereof, as compared to the native production of, or the enzymatic activity of, the polypeptide. This is done by the means of two methods—either by placing the nucleotide under a stronger promoter OR by having multiple copies of the necessary gene inserted in the chromosome of the fermenting organism.

The terms “activity,” “activities,” “enzymatic activity,” and “enzymatic activities” are used interchangeably and are intended to include any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof. Techniques for determining total activity as compared to secreted activity are described herein and are known in the art

The term “secreted” is intended to include the movement of polypeptides to the periplasmic space or extracellular milieu. The term “increased secretion” is intended to include situations in which a given polypeptide is secreted at an increased level (i.e., in excess of the naturally occurring amount of secretion). In certain embodiments, the term “increased secretion” refers to an increase in secretion of a given polypeptide as compared to the naturally occurring level of secretion.

The term “secretory polypeptide” is intended to include any polypeptide(s), alone or in combination with other polypeptides, that facilitate the transport of another polypeptide from the intracellular space of a cell to the extracellular milieu. In certain embodiments, the secretory polypeptide(s) encompass all the necessary secretory polypeptides sufficient to impart secretory activity to a Gram-negative or Gram-positive host cell or to a yeast host cell. Typically, secretory proteins are encoded in a single region or locus that may be isolated from one host cell and transferred to another host cell using genetic engineering. In certain embodiments, the secretory polypeptide(s) are derived from any bacterial cell having secretory activity or any yeast cell having secretory activity. In certain embodiments, the secretory polypeptide(s) are derived from a host cell having Type II secretory activity. In certain embodiments, the host cell is a thermophilic bacterial cell. In certain embodiments, the host cell is a yeast cell.

The term “derived from” is intended to include the isolation (in whole or in part) of a polynucleotide segment from an indicated source or the purification of a polypeptide from an indicated source. The term is intended to include, for example, direct cloning, PCR amplification, or artificial synthesis from or based on a sequence associated with the indicated polynucleotide source.

Certain embodiments of the present invention provide for the “insertion,” (e.g., the addition, integration, incorporation, or introduction) of certain genes or particular polynucleotide sequences within thermophilic or mesophilic microorganisms, which insertion of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

In certain embodiments, the polynucleotide sequences of the invention are “genetically modified” such that the encoded enzyme is engineered to alter catalytic activity and/or alter substrate specificity to improve the conversion of a substrate to a product as compared to the native enzyme. In certain aspects, the “genetic modification” alters catalytic activity and/or substrate specificity to provide an encoded enzyme that converts a substrate to a product that is not catalyzed by the native enzyme in vivo, or is catalyzed at only minimal turnover. Techniques to genetically modify polynucleotides are known in the art and include, but are not limited to, alteration, insertion, and/or deletion of one or more nucleic acids in the polynucleotide. Such techniques to alter, insert, and/or delete nucleic acids include, but are not limited to, random, site-directed, or saturating mutagenesis.

Certain embodiments of the present invention provide for the “inactivation” or “deletion” of certain genes or particular polynucleotide sequences within thermophilic or mesophilic microorganisms, which “inactivation” or “deletion” of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

The term “bioprocessing” is intended to include a processing strategy for cellulosic biomass or starch-based biomass or molasses-based biomass that involves consolidating into a single process step, four biologically-mediated events: enzyme production, hydrolysis, hexose fermentation, and pentose fermentation. Implementing this strategy requires development of microorganisms that both utilize cellulose, hemicelluloses, and other biomass components while also producing a product of interest at sufficiently high yield and concentrations.

In one aspect of the invention, the genes or particular polynucleotide sequences are inserted to activate the activity for which they encode, such as the expression of an enzyme. In certain embodiments, genes encoding enzymes in the metabolic production of fatty acids may be added to a mesophilic or a thermophilic organism. In one aspect of the invention, the genes or particular polynucleotide

sequences are partially, substantially, or completely deleted, silenced, inactivated, or down-regulated in order to inactivate the activity for which they encode, such as the expression of an enzyme. Deletions provide maximum stability because there is no opportunity for a reverse mutation to restore function. Alternatively, genes can be partially, substantially, or completely deleted, silenced, inactivated, or down-regulated by insertion of nucleic acid sequences that disrupt the function and/or expression of the gene (e.g., P1 transduction or other methods known in the art). The terms “eliminate,” “elimination,” and “knockout” are used interchangeably with the terms “deletion,” “partial deletion,” “substantial deletion,” or “complete deletion.” In certain embodiments, strains of thermophilic or mesophilic microorganisms of interest may be engineered by site directed homologous recombination to knockout the production of organic acids. In still other embodiments, RNAi or antisense DNA (asDNA) may be used to partially, substantially, or completely silence, inactivate, or down-regulate a particular gene of interest.

In certain embodiments, the genes targeted for deletion or inactivation as described herein may be endogenous to the native strain of the microorganism, and may thus be understood to be referred to as “native gene(s)” or “endogenous gene(s).”

An organism is in “a native state” if it has not been genetically engineered or otherwise manipulated by the hand of man in a manner that intentionally alters the genetic and/or phenotypic constitution of the organism. For example, wild-type organisms may be considered to be in a native state. In other embodiments, the gene(s) targeted for deletion or inactivation may be non-native to the organism.

Similarly, the enzymes of the invention as described herein can be endogenous to the native strain of the microorganism, and can thus be understood to be referred to as “native” or “endogenous.”

The term “upregulated” means increased in activity, e.g., increase in enzymatic activity of the enzyme as compared to activity in a native host organism.

The term “downregulated” means decreased in activity, e.g., decrease in enzymatic activity of the enzyme as compared to activity in a native host organism.

The term “activated” means expressed or metabolically functional.

As used herein, the term “Essential Amino Acids” is intended to include those amino acids which are known not to be produced by mammals, which they have to acquire through diet. They are histidine, isoleucine, leucine, lysine, methionine, phenylalanine, threonine, tryptophan and valine.

As used herein, the term “High protein rich animal feed” is intended to include an animal (cattle, pig, poultry, fish) feed with protein content greater than or equal to 35% with predominance of these above-mentioned essential amino acids.

As used herein, the term “alcohol” is intended to include compounds formed by the addition of one or more hydroxyl functional group (—OH) a hydrocarbon. Examples of alcohol derivatives include, but are not limited to, ethanol, methanol, butanol, medium-chain fatty alcohols, branched alcohols, poly-ols etc. As used herein, the term “lipids” is intended to include compounds formed

by the addition of one or more carboxyl functional group (—OH) a hydrocarbon or the derivates such as ether, ester groups. Examples of lipids include, but are not limited to, short-chain free fatty acids (C4 to C8), medium-chain free fatty acids (C8 to C16), long-chain free fatty acids (C16 and above), unsaturated fatty acids, triglycerides, fatty-esters, branched fatty acids, poly-carboxylic fatty acids etc.

The term “carbohydrate source” is intended to include any source of carbohydrate including, but not limited to, biomass or carbohydrates, such as a sugar or a sugar alcohol. “Carbohydrates” include, but are not limited to, monosaccharides (e.g., glucose, fructose, galactose, xylose, arabinose, or ribose), sugar derivatives (e.g., sorbitol, maltose, or lactose), oligosaccharides (e.g., xylooligomers, cellodextrins, or maltodextrins), and polysaccharides (e.g., xylan, cellulose, hemicellulose, starch, mannan, alginate, or pectin).

As used herein, the term “anaerobic” is intended to include conditions in which oxygen or air is not supplied to the organism during the bioprocess. An anaerobic organism is one that does not require for growth. Anaerobic conditions include those in which an optional terminal electron acceptor such as nitrates, nitrites, chlorates, perchlorates, fumarate, hypochlourous ions is provided during fermentation process (either throughout or in phases).

The term “Metabolic Pathway” describes the biochemical steps within a microorganism that enables it to anaerobically ferment simple hexose sugars into a combination of acidic and pH-neutral products via the method of glycolysis. The glycolytic pathway is abundant and comprises a series of enzymatic steps whereby a six-carbon glucose molecule is counteracted, via multiple intermediates, into two molecules of the three-carbon compound pyruvate. This process leads to the online generation of ATP (biological energy supply) and therefore the reduced cofactor NADH.

Pyruvate is a very important intermediary compound of metabolism that can be oxidized to acetyl coenzyme A (acetyl CoA), which then enters the tricarboxylic acid cycle (TCA), which successively generates synthetic precursors, CO2 and reduced cofactors. The cofactors are then oxidized by donating hydrogen equivalents, via a series of enzymatic steps, to oxygen leading to the formation of water and ATP. This process of energy formation is thought of as a biological process.

Under anaerobic conditions (no available oxygen), fermentation occurs during which the degradation products of organic compounds function hydrogen donors and acceptors. Excess NADH from glycolysis is oxidized in reactions involving the reduction of organic substrates to products, like lactate and ethanol. Additionally, ATP is regenerated from the assembly of organic acids, like acetate, in a very process referred to as substrate level phosphorylation. Therefore, the fermentation products of glycolysis and pyruvate metabolism include a spread of organic acids, alcohols and CO2.

The term “Biomass” can include any variety of biomass known within the art or described herein. The terms “lignocellulosic material,” “lignocellulosic substrate,” and “cellulosic biomass” mean any variety of biomass comprising cellulose, hemicellulose, lignin, or combinations thereof, like but not limited to woody biomass, forage grasses, herbaceous energy crops, non-woody-plant biomass, agricultural wastes and/or agricultural residues, forestry residues and/or forestry wastes, paper-production sludge and/or paper sludge, waste-water-treatment sludge, municipal solid waste, corn fiber from wet and dry mill corn ethanol plants, and sugar-processing residues.

The terms “hemicellulosics,” “hemicellulosic portions,” and “hemicellulosic fractions” mean the non-lignin, non-cellulose elements of lignocellulosic material, like but not limited to hemicellulose (i.e., comprising xyloglucan, xylan, glucuronoxylan, arabinoxylan, mannan, glucomannan, and galactoglucomannan), pectins (e.g., homogalacturonans, rhamnogalacturonan I and II, and xylogalacturonan), and proteoglycans (e.g., arabinogalactan-protein, extensin, and proline-rich proteins).

The term “lignocellulosic material” can include, but isn't limited to, woody biomass, like recycled pulp fiber, sawdust, hardwood, softwood, and combinations thereof; grasses, like panic grass, cord grass, rye grass, reed birdseed grass, miscanthus, or a mix thereof; sugar-processing residues, like but not limited to sugar cane bagasse; agricultural wastes, like but not limited to rice straw, rice hulls, barley straw, corn cobs, cereal straw, wheat straw, canola straw, oat straw, oat hulls, and corn fiber; stover, like but not limited to soybean stover, corn stover; succulents, like but not limited to, Agave; and forestry wastes, like but not limited to, recycled pulp fiber, sawdust, hardwood (e.g., poplar, oak, maple, birch, willow), softwood, or any combination thereof. “Lignocellulosic material” may comprise one species of fiber; alternatively, lignocellulosic material may comprise a mix of fibers that originate from different lignocellulosic materials. Other “lignocellulosic materials” are agricultural wastes, like cereal straws, including wheat straw, barley straw, canola straw and oat straw; corn fiber; stovers, like corn stover and soybean stover; grasses, like panic grass, reed grass, cord grass, and miscanthus; or combinations thereof.

The term “vector(s)” relates to vectors which include genes encoding enzymes of the invention, as described above, moreover as host cells which are genetically engineered with vectors of the invention and therefore the production of polypeptides of the invention by recombinant techniques. A DNA sequence inserted within the vector is operatively linked to an appropriate expression control sequence(s) (promoter) to direct mRNA synthesis. Any suitable promoter to drive gene expression within the host cells of the invention may be used. Additionally, promoters known to regulate expression of genes in prokaryotic or lower eukaryotic cells may be used. The expression vector also contains a ribosome binding site for translation initiation and a transcription terminator. The vector may also include appropriate sequences for amplifying expression, or can include additional regulatory regions.

The vector containing a suitable selectable marker sequence and an appropriate promoter or control sequence, is employed to engineer an appropriate thermophilic host to allow the host to express a specific protein.

The term “host cell(s)” refers to cell(s) that may be genetically engineered (transduced or transformed or transfected) with the vector(s) of this invention which might be, as an example, a cloning vector or an expression vector. The vector will be, as an example, within the type of a plasmid, a viral particle, a phage, etc. The engineered host cells are cultured in conventional nutrient media modified as appropriate for activating promoters, selecting transformants or amplifying the genes of the current invention. The culture conditions, like temperature, pH and also the like, are those previously used with the host cell selected for expression, and can be apparent to the ordinarily skilled artisan.

Host cells useful within the present invention include any prokaryotic or eukaryotic cells; as an example, microorganisms selected from bacterial, algal, and yeast cells. Among host cells thus suitable for this invention are microorganisms, for instance, of the genera Aeromonas, Aspergillus, Bacillus, Escherichia, Kluyveromyces, Pichia, Rhodococcus, Saccharomyces and Streptomyces.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

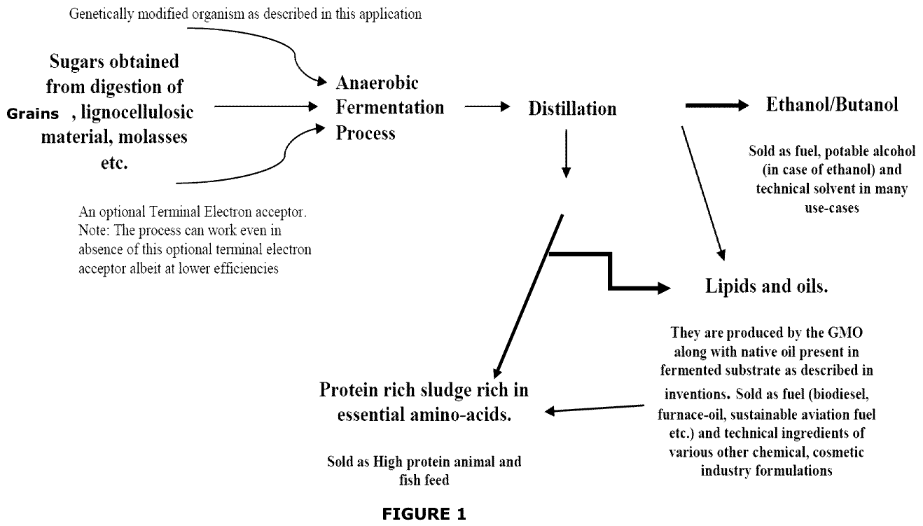

FIG. 1. Overview of the added-value products produced by the genetically-modified microorganism and anaerobic fermentation process according to the invention.

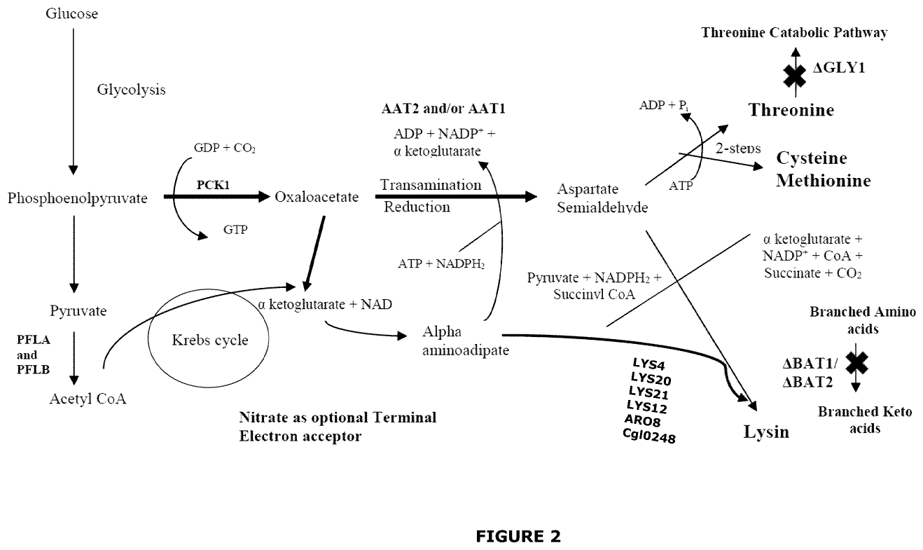

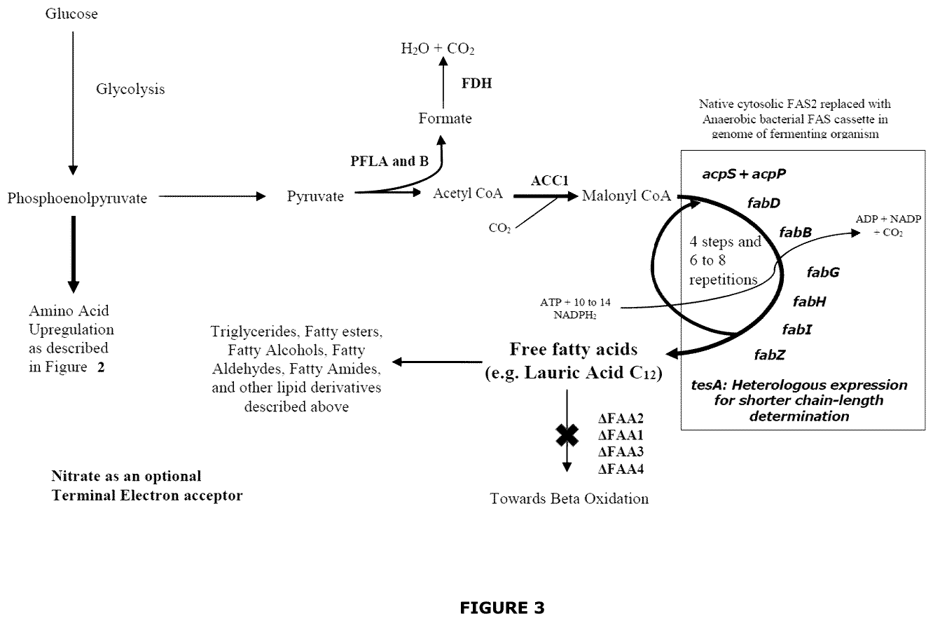

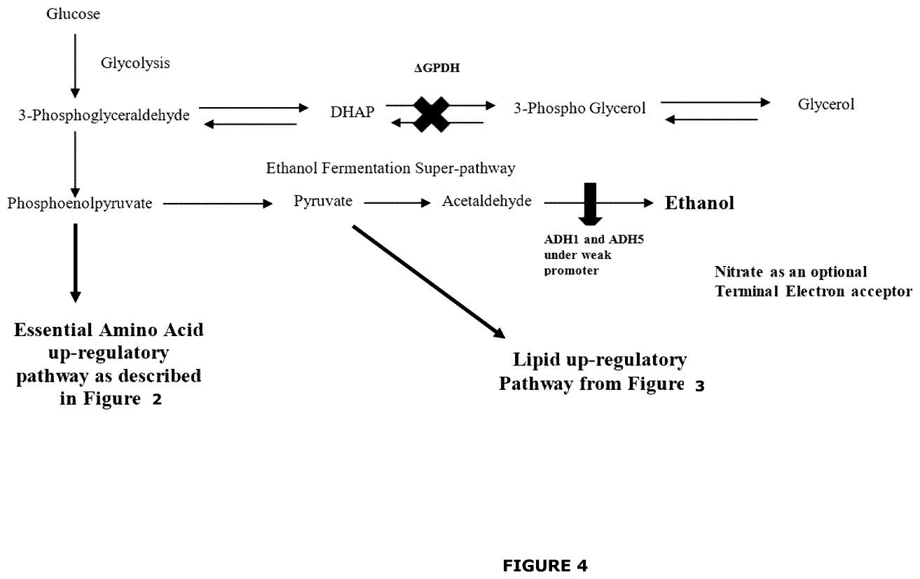

FIG. 2. Essential amino acid biosynthesis pathway upregulation scheme. A bold arrows represent upregulation of a metabolic pathway in the genetically modified microorganism of the invention. A cross represents genetic downregulation of a catabolic pathway for production and secretion of a metabolic product.

FIG. 3. Anaerobic lipid co-production pathway upregulation scheme. A bold arrow indicates a gene that is overexpressed (a native or heterologously expressed gene). A crossed pathway indicates downregulation of expression of a gene either by gene-deletion or placing the gene under the control of a weak promotor.

FIG. 4. Anaerobic pathways for the co-production of alcohol with essential amino-acids and lipids in the genetically modified microorganism of the invention.

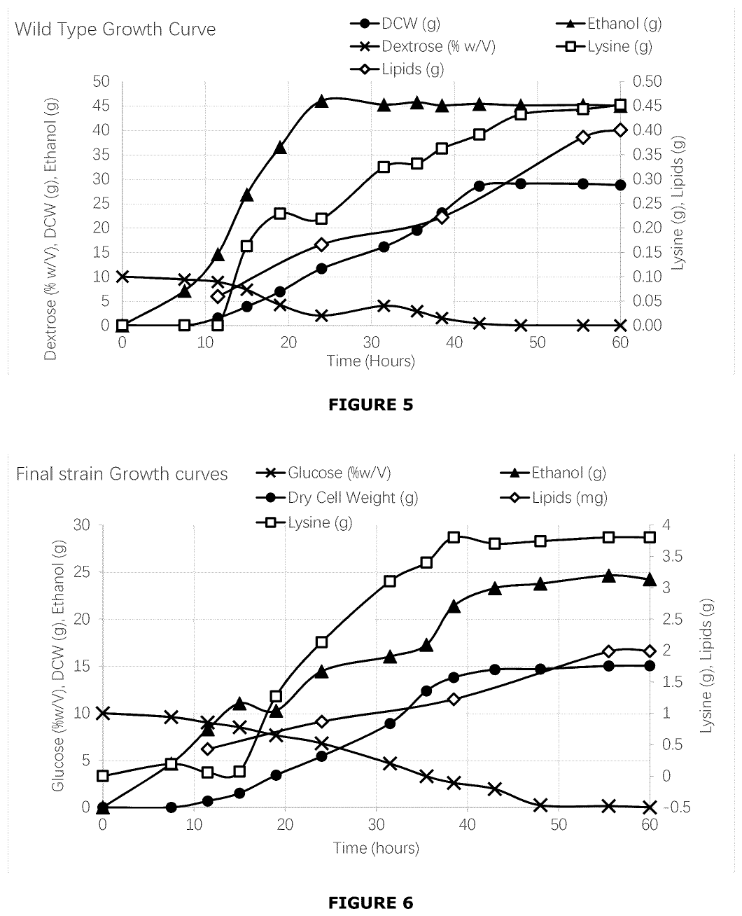

FIG. 5. A graph showing the growth curve of a parent wild-type yeast strain, measured as dry cell weight (DCW), during anaerobic fermentation growth on Yeast Nitrogen Base (YNB) defined media, further showing the profile of dextrose consumption and lysine, ethanol, and extracellular lipid secreted by the yeast strain. The unmodified strain was grown on (YNB) medium on one liter working volume in erlenmeyer conical flasks of 5 liter volume in triplicates. Ergo

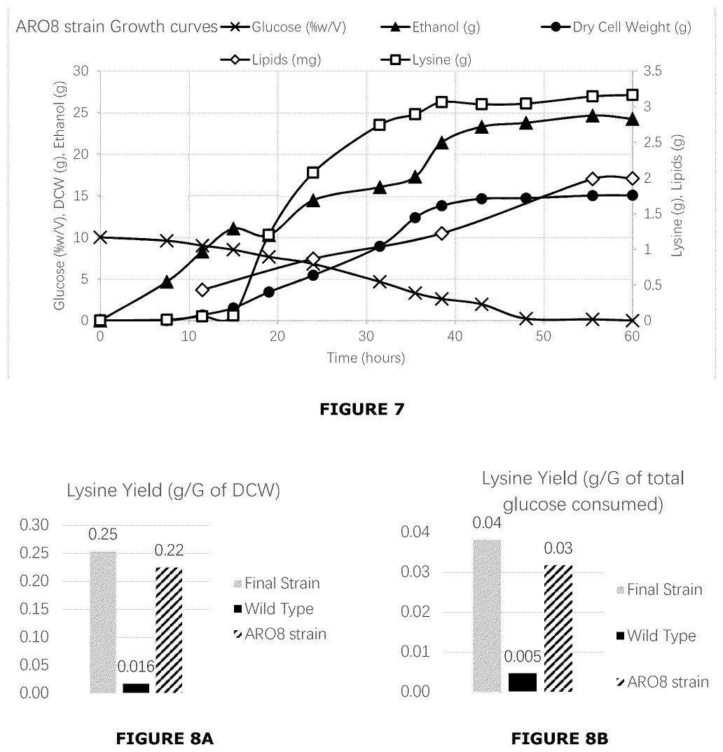

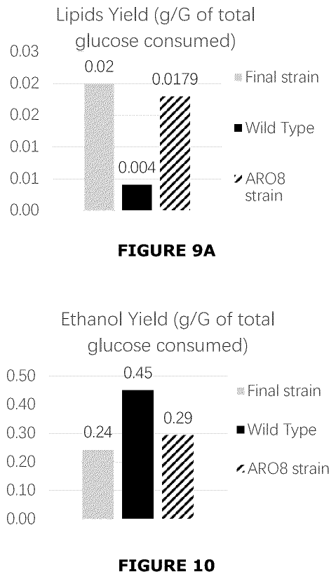

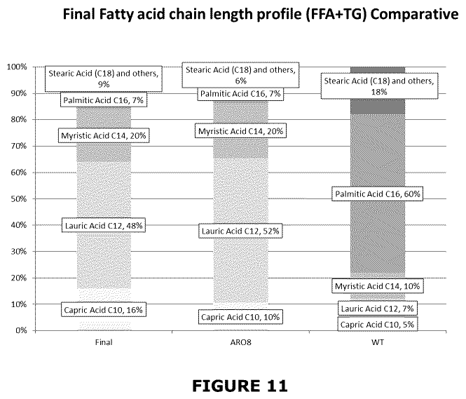

FIG. 6. A graph showing the growth curve of a genetically modified yeast strain (Final strain) of the invention, measured as dry cell weight (DCW), during anaerobic fermentation growth on a YNB defined media, further showing the profile of dextrose consumption and lysine, ethanol, and extracellular lipid secreted by the yeast strain. Final Strain Genotype: Δfaa2, Δfaa2, Δfaa1, Δfaa4+PACC1::PTEF1+PFLA+PFLB+Bacterial FAS Cassette+Palm Thioesterase::Yeast Thioesterase+PADH::PNADHKinase+Bacterially Krebs Cycle genes+ΔGPDH1+ΔADH1+ΔADH5+ΔGLY1+PGPD1::PAAT1+bacterial PCK1+PLYS20::PCYCl+PADH2::PLYS4+Lys20+PLYS12::PZEV1+PLYS21::PPGKl+C gl0248

FIG. 7. A graph showing the growth curve of a genetically modified yeast strain (ARO8 strain) of the invention, measured as dry cell weight (DCW), during anaerobic fermentation growth on a YNB defined media, further showing the profile of dextrose consumption and lysine, ethanol, and extracellular lipid secreted by the yeast strain. ARO8 Strain Genotype:
Δfaa2+Δfaa3+Δfaa1+PACC1 ::PTEF1+ADH5::PFLA_AND_PFLB+Yeast_Thioesterase::C uphea wrightii Thioesterase+GPD2::Bacterial FAS Cassette+PNADKinase::PADH1+ADH1::Bacterial Krebs Cycle Genes Cassette+ΔGPD2+ΔADH1+ΔADH5+ΔGLY1+PAAT1::PGPD1+YeastPCK1::bacterial PCK1+PLYS20::PCYC1+PLYS4::PADH1+PLYS12 ::PZEV1-2+FAA1::cg10248+FAA3::PP3:AR08

FIG. 8. Histogram showing a comparison of the lysine yield of a parent wild-type yeast strain and genetically modified yeast strains (ARO8 and Final strain) derived from the parent strain, expressed as A: grams lysine per gram of yeast dry cell weight and B: grams lysine per gram glucose consumed,.

FIG. 9. Histogram showing a comparison of the lipid yield of a parent wild-type yeast strain and genetically modified yeast strains (ARO8 and Final strain) derived from the parent strain, expressed as grams total lipids [comprising free fatty acids and triglycerides] per gram of glucose consumed.

FIG. 10. Histogram showing a comparison of the ethanol yield of a parent wild-type yeast strain and genetically modified yeast strains (ARO8 and Final strain) derived from the parent strain, expressed as grams total ethanol per gram of glucose consumed.

FIG. 11. Histogram showing comparison of final fatty acid chain-length profile of lipids (Free fatty acids+Triglycerides) produced by parent wild-type yeast strain and genetically modifiec yeast strains (ARO8 and Final strain) derived from the parent strain, expressed as percentage of total lipids (free fatty acids and triglycerides) produced by the respective strains.

### Definitions

The indefinite articles “a” and “an” preceding an element or component of the invention are intended to include plurals of the element or component, e.g., one or at least one of the element or components, unless the context is such that only the singular form is intended.

The term “heterologous” when used in reference to a polynucleotide, a gene, a polypeptide, or an enzyme refers to a polynucleotide, gene, polypeptide, or an enzyme not normally found in the host organism. “Heterologous” also includes a native coding region, or portion thereof, that is reintroduced into the source organism in a form that is different from the corresponding native gene, e.g., not in its natural location in the organism's genome. The heterologous polynucleotide or gene may be introduced into the host organism by, e.g., gene transfer. A heterologous gene may include a native coding region that is a portion of a chimeric gene including non-native regulatory regions that is reintroduced into the native host. Foreign genes can comprise native genes inserted into a non-native organism, or chimeric genes.

The term “heterologous polynucleotide” is intended to include a polynucleotide that encodes one or more polypeptides or portions or fragments of polypeptides. A heterologous polynucleotide may be derived from any source, e.g., eukaryotes, prokaryotes, viruses, or synthetic polynucleotide fragments.

The terms “promoter” or “surrogate promoter” is intended to include a polynucleotide that can transcriptionally control a gene-of-interest that it does not transcriptionally control in nature. In certain embodiments, the transcriptional control of a surrogate promoter results in an increase in expression of the gene-of-interest. In certain embodiments, a surrogate promoter is placed 5′ to the gene-of-interest. A surrogate promoter may be used to replace the natural promoter, or may be used in addition to the natural promoter. A surrogate promoter may be endogenous with regard to the host cell in which it is used, or it may be a heterologous polynucleotide sequence introduced into the host cell, e.g., exogenous with regard to the host cell in which it is used.

The term's “gene(s)” or “polynucleotide” or “polynucleotide sequence(s)” are intended to include nucleic acid molecules, e.g., polynucleotides which include an open reading frame encoding a polypeptide, and can further include non-coding regulatory sequences, and introns. In addition, the terms are intended to include one or more genes that map to a functional locus. In addition, the terms are intended to include a specific gene for a selected purpose. The gene may be endogenous to the host cell or may be recombinantly introduced into the host cell, e.g., as a plasmid maintained episomally or a plasmid (or fragment thereof) that is stably integrated into the genome. In addition to the plasmid form, a gene may, for example, be in the form of linear DNA. The term gene is also intended to cover all copies of a particular gene, e.g., all of the DNA sequences in a cell encoding a particular gene product.

The term “transcriptional control” is intended to include the ability to modulate gene expression at the level of transcription. In certain embodiments, transcription, and thus gene expression, is modulated by replacing or adding a surrogate promoter near the end of the coding region of a gene-of-interest, thereby resulting in altered gene expression. In certain embodiments, the transcriptional control of one or more genes is engineered to result in the optimal expression of such genes, e.g., in a desired ratio. The term also includes inducible transcriptional control as recognized in the art.

The term “expression” is intended to include the expression of a gene at least at the level of mRNA production.

The term “expression product” is intended to include the resultant product, e.g., a polypeptide, of an expressed gene.

The term “polypeptide” is intended to encompass a singular “polypeptide,” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the amino acids. Thus, peptides, dipeptides, tripeptides, oligopeptides, “protein,” “amino acid chain,” “enzyme,” or any other term used to refer to a chain or chains of two or more amino acids, are included within the definition of “polypeptide,” and the term “polypeptide” may be used instead of, or interchangeably with, any of these terms. A polypeptide may be derived from a natural biological source or produced by recombinant technology. It may be generated in any manner, including by chemical synthesis.

The term “increased expression” is intended to include an alteration in gene expression at least at the level of increased mRNA production and, preferably, at the level of polypeptide expression. The term “increased production” is intended to include an increase in the amount of a polypeptide expressed, in the level of the enzymatic activity of the polypeptide, or a combination thereof, as compared to the native production of, or the enzymatic activity of, the polypeptide. This is done by the means of two methods — either by placing the nucleotide under a stronger promoter OR by having multiple copies of the necessary gene inserted in the chromosome of the fermenting organism.

The terms “activity,” “activities,” “enzymatic activity,” and “enzymatic activities” are used interchangeably and are intended to include any functional activity normally attributed to a selected polypeptide when produced under favorable conditions.

Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof. Techniques for determining total activity as compared to secreted activity are described herein and are known in the art

The term “secreted” is intended to include the movement of polypeptides to the periplasmic space or extracellular milieu. The term “increased secretion” is intended to include situations in which a given polypeptide is secreted at an increased level (i.e., in excess of the naturally occurring amount of secretion). In certain embodiments, the term “increased secretion” refers to an increase in secretion of a given polypeptide as compared to the naturally occurring level of secretion.

The term “secretory polypeptide” is intended to include any polypeptide(s), alone or in combination with other polypeptides, that facilitate the transport of another polypeptide from the intracellular space of a cell to the extracellular milieu. In certain embodiments, the secretory polypeptide(s) encompass all the necessary secretory polypeptides sufficient to impart secretory activity to a Gram-negative or Gram-positive host cell or to a yeast host cell. Typically, secretory proteins are encoded in a single region or locus that may be isolated from one host cell and transferred to another host cell using genetic engineering. In certain embodiments, the secretory polypeptide(s) are derived from any bacterial cell having secretory activity or any yeast cell having secretory activity. In certain embodiments, the secretory polypeptide(s) are derived from a host cell having Type II secretory activity. In certain embodiments, the host cell is a thermophilic bacterial cell. In certain embodiments, the host cell is a yeast cell.

The term “derived from” is intended to include the isolation (in whole or in part) of a polynucleotide segment from an indicated source or the purification of a polypeptide from an indicated source. The term is intended to include, for example, direct cloning, PCR amplification, or artificial synthesis from or based on a sequence associated with the indicated polynucleotide source.

Certain embodiments of the present invention provide for the “insertion,” (e.g., the addition, integration, incorporation, or introduction) of certain genes or particular polynucleotide sequences within thermophilic or mesophilic microorganisms, which insertion of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

In certain embodiments, the polynucleotide sequences of the invention are “genetically modified” such that the encoded enzyme is engineered to alter catalytic activity and/or alter substrate specificity to improve the conversion of a substrate to a product as compared to the native enzyme. In certain aspects, the “genetic modification” alters catalytic activity and/or substrate specificity to provide an encoded enzyme that converts a substrate to a product that is not catalyzed by the native enzyme in vivo, or is catalyzed at only minimal turnover. Techniques to genetically modify polynucleotides are known in the art and include, but are not limited to, alteration, insertion, and/or deletion of one or more nucleic acids in the polynucleotide. Such techniques to alter, insert, and/or delete nucleic acids include, but are not limited to, random, site-directed, or saturating mutagenesis.

Certain embodiments of the present invention provide for the “inactivation” or “deletion” of certain genes or particular polynucleotide sequences within thermophilic or mesophilic microorganisms, which “inactivation” or “deletion” of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

The term “bioprocessing” is intended to include a processing strategy for cellulosic biomass or starch-based biomass or molasses-based biomass that involves consolidating into a single process step, four biologically-mediated events: enzyme production, hydrolysis, hexose fermentation, and pentose fermentation. Implementing this strategy requires development of microorganisms that both utilize cellulose, hemicelluloses, and other biomass components while also producing a product of interest at sufficiently high yield and concentrations.

In one aspect of the invention, the genes or particular polynucleotide sequences are inserted to activate the activity for which they encode, such as the expression of an enzyme. In certain embodiments, genes encoding enzymes in the metabolic production of fatty acids may be added to a mesophilic or a thermophilic organism.

In one aspect of the invention, the genes or particular polynucleotide sequences are partially, substantially, or completely deleted, silenced, inactivated, or down-regulated in order to inactivate the activity for which they encode, such as the expression of an enzyme. Deletions provide maximum stability because there is no opportunity for a reverse mutation to restore function. Alternatively, genes can be partially, substantially, or completely deleted, silenced, inactivated, or down-regulated by insertion of nucleic acid sequences that disrupt the function and/or expression of the gene (e.g., P1 transduction or other methods known in the art). The terms “eliminate,” “elimination,” and “knockout” are used interchangeably with the terms “deletion,” “partial deletion,” “substantial deletion,” or “complete deletion.” In certain embodiments, strains of thermophilic or mesophilic microorganisms of interest may be engineered by site directed homologous recombination to knockout the production of organic acids. In still other embodiments, RNAi or antisense DNA (asDNA) may be used to partially, substantially, or completely silence, inactivate, or down-regulate a particular gene of interest.

In certain embodiments, the genes targeted for deletion or inactivation as described herein may be endogenous to the native strain of the microorganism, and may thus be understood to be referred to as “native gene(s)” or “endogenous gene(s).”

An organism is in “a native state” if it has not been genetically engineered or otherwise manipulated by the hand of man in a manner that intentionally alters the genetic and/or phenotypic constitution of the organism. For example, wild-type organisms may be considered to be in a native state. In other embodiments, the gene(s) targeted for deletion or inactivation may be non-native to the organism.

Similarly, the enzymes of the invention as described herein can be endogenous to the native strain of the microorganism, and can thus be understood to be referred to as “native” or “endogenous.”

The term “upregulated” means increased in activity, e.g., increase in enzymatic activity of the enzyme as compared to activity in a native host organism.

The term “downregulated” means decreased in activity, e.g., decrease in enzymatic activity of the enzyme as compared to activity in a native host organism.

The term “activated” means expressed or metabolically functional.

As used herein, the term “Essential Amino Acids” is intended to include those amino acids which are known not to be produced by mammals, which they have to acquire through diet. They are histidine, isoleucine, leucine, lysine, methionine, phenylalanine, threonine, tryptophan and valine.

As used herein, the term “High protein rich animal feed” is intended to include an animal (cattle, pig, poultry, fish) feed with protein content greater than or equal to 35% with predominance of these above-mentioned essential amino acids.

As used herein, the term “alcohol” is intended to include compounds formed by the addition of one or more hydroxyl functional group (—OH) a hydrocarbon. Examples of alcohol derivatives include, but are not limited to, ethanol, methanol, butanol, medium-chain fatty alcohols, branched alcohols, poly-ols etc. As used herein, the term “lipids” is intended to include compounds formed

by the addition of one or more carboxyl functional group (—OH) a hydrocarbon or the derivates such as ether, ester groups. Examples of lipids include, but are not limited to, short-chain free fatty acids (C4 to C8), medium-chain free fatty acids (C8 to C16), long-chain free fatty acids (C16 and above), unsaturated fatty acids, triglycerides, fatty-esters, branched fatty acids, poly-carboxylic fatty acids etc.

The term “carbohydrate source” is intended to include any source of carbohydrate including, but not limited to, biomass or carbohydrates, such as a sugar or a sugar alcohol. “Carbohydrates” include, but are not limited to, monosaccharides (e.g., glucose, fructose, galactose, xylose, arabinose, or ribose), sugar derivatives (e.g., sorbitol, maltose, or lactose), oligosaccharides (e.g., xylooligomers, cellodextrins, or maltodextrins), and polysaccharides (e.g., xylan, cellulose, hemicellulose, starch, mannan, alginate, or pectin).

As used herein, the term “anaerobic” is intended to include conditions in which oxygen or air is not supplied to the organism during the bioprocess. An anaerobic organism is one that does not require for growth. Anaerobic conditions include those in which an optional terminal electron acceptor such as nitrates, nitrites, chlorates, perchlorates, fumarate, hypochlourous ions is provided during fermentation process (either throughout or in phases).

The term “Metabolic Pathway” describes the biochemical steps within a microorganism that enables it to anaerobically ferment simple hexose sugars into a combination of acidic and pH-neutral products via the method of glycolysis. The glycolytic pathway is abundant and comprises a series of enzymatic steps whereby a six-carbon glucose molecule is counteracted, via multiple intermediates, into two molecules of the three-carbon compound pyruvate. This process leads to the online generation of ATP (biological energy supply) and therefore the reduced cofactor NADH.

Pyruvate is a very important intermediary compound of metabolism that can be oxidized to acetyl coenzyme A (acetyl CoA), which then enters the tricarboxylic acid cycle (TCA), which successively generates synthetic precursors, CO2 and reduced cofactors. The cofactors are then oxidized by donating hydrogen equivalents, via a series of enzymatic steps, to oxygen leading to the formation of water and ATP. This process of energy formation is thought of as a biological process.

Under anaerobic conditions (no available oxygen), fermentation occurs during which the degradation products of organic compounds function hydrogen donors and acceptors. Excess NADH from glycolysis is oxidized in reactions involving the reduction of organic substrates to products, like lactate and ethanol. Additionally, ATP is regenerated from the assembly of organic acids, like acetate, in a very process referred to as substrate level phosphorylation. Therefore, the fermentation products of glycolysis and pyruvate metabolism include a spread of organic acids, alcohols and CO2.

The term “Biomass” can include any variety of biomass known within the art or described herein. The terms “lignocellulosic material,” “lignocellulosic substrate,” and “cellulosic biomass” mean any variety of biomass comprising cellulose, hemicellulose, lignin, or combinations thereof, like but not limited to woody biomass, forage grasses, herbaceous energy crops, non-woody-plant biomass, agricultural wastes and/or agricultural residues, forestry residues and/or forestry wastes, paper-production sludge and/or paper sludge, waste-water-treatment sludge, municipal solid waste, corn fiber from wet and dry mill corn ethanol plants, and sugar-processing residues.

The terms “hemicellulosics,” “hemicellulosic portions,” and “hemicellulosic fractions” mean the non-lignin, non-cellulose elements of lignocellulosic material, like but not limited to hemicellulose (i.e., comprising xyloglucan, xylan, glucuronoxylan, arabinoxylan, mannan, glucomannan, and galactoglucomannan), pectins (e.g., homogalacturonans, rhamnogalacturonan I and II, and xylogalacturonan), and proteoglycans (e.g., arabinogalactan-protein, extensin, and proline-rich proteins).

The term “lignocellulosic material” can include, but isn't limited to, woody biomass, like recycled pulp fiber, sawdust, hardwood, softwood, and combinations thereof; grasses, like panic grass, cord grass, rye grass, reed birdseed grass, miscanthus, or a mix thereof; sugar-processing residues, like but not limited to sugar cane bagasse; agricultural wastes, like but not limited to rice straw, rice hulls, barley straw, corn cobs, cereal straw, wheat straw, canola straw, oat straw, oat hulls, and corn fiber; stover, like but not limited to soybean stover, corn stover; succulents, like but not limited to, Agave; and forestry wastes, like but not limited to, recycled pulp fiber, sawdust, hardwood (e.g., poplar, oak, maple, birch, willow), softwood, or any combination thereof. “Lignocellulosic material” may comprise one species of fiber; alternatively, lignocellulosic material may comprise a mix of fibers that originate from different lignocellulosic materials. Other “lignocellulosic materials” are agricultural wastes, like cereal straws, including wheat straw, barley straw, canola straw and oat straw; corn fiber; stovers, like corn stover and soybean stover; grasses, like panic grass, reed grass, cord grass, and miscanthus; or combinations thereof.

The term “vector(s)” relates to vectors which include genes encoding enzymes of the invention, as described above, moreover as host cells which are genetically engineered with vectors of the invention and therefore the production of polypeptides of the invention by recombinant techniques. A DNA sequence inserted within the vector is operatively linked to an appropriate expression control sequence(s) (promoter) to direct mRNA synthesis. Any suitable promoter to drive gene expression within the host cells of the invention may be used. Additionally, promoters known to regulate expression of genes in prokaryotic or lower eukaryotic cells may be used. The expression vector also contains a ribosome binding site for translation initiation and a transcription terminator. The vector may also include appropriate sequences for amplifying expression, or can include additional regulatory regions.

The vector containing a suitable selectable marker sequence and an appropriate promoter or control sequence, is employed to engineer an appropriate thermophilic host to allow the host to express a specific protein.

The term “host cell(s)” refers to cell(s) that may be genetically engineered (transduced or transformed or transfected) with the vector(s) of this invention which might be, as an example, a cloning vector or an expression vector. The vector will be, as an example, within the type of a plasmid, a viral particle, a phage, etc. The engineered host cells are cultured in conventional nutrient media modified as appropriate for activating promoters, selecting transformants or amplifying the genes of the current invention. The culture conditions, like temperature, pH and also the like, are those previously used with the host cell selected for expression, and can be apparent to the ordinarily skilled artisan.

Host cells useful within the present invention include any prokaryotic or eukaryotic cells; as an example, microorganisms selected from bacterial, algal, and yeast cells. Among host cells thus suitable for this invention are microorganisms, for instance, of the genera Aeromonas, Aspergillus, Bacillus, Escherichia, Kluyveromyces, Pichia, Rhodococcus, Saccharomyces and Streptomyces.

### DETAILED DESCRIPTION OF THE INVENTION

The recombinant microorganisms and methods of the invention use metabolic pathways that allow for the anaerobic co-production of essential amino acids (such as Lysine, Threonine etc) which humans and other higher mammals cannot synthesize de novo along with alcohol and malonyl-CoA derived products such as lipids (see FIG. 1). The metabolic pathways allow for the co-production of these aforementioned compounds along with lipid based long chain compounds, including, e.g., chain lengths from 2 carbon atoms up to 20 carbon atoms per molecule, and cellular growth in the absence of oxygen or other mechanisms to generate cellular energy (ATP) besides fermentative metabolism.

An aspect of the invention is the ability to co-produce essential amino-acids and alcohol with an anaerobic process rather than with an aerobic process. Anaerobic production results in easier scalability and optimum process thermodynamics. For bio-conversion of carbohydrate-based substrates to low-cost-high-volume products, an anaerobic process is even more desirable, as the requirement for oxygen transfer in a medium with suspended solids is highly unattractive from an engineering perspective. Few more advantages which contribute to diversification of products produced by a manufacturer along with improvement in the bottom-line include, but are not limited to:
1) Co-Production of a high-protein animal feed which is rich in essential amino acids thereby contributing to the value of dairy, meat and animal husbandry industry;
2) Lower separation costs from a dilute aqueous fermentation as a result of the immiscible nature of long chain lipids compared to fully miscible shorter chain compounds;
3) Greater downstream product diversity and flexibility; and

One aspect of the invention relates to a recombinant microorganism comprising one or more native and/or heterologous enzymes that function in one or more engineered metabolic pathways to convert a carbohydrate source to an essential amino acids and lipids, wherein the one or more native and/or heterologous enzymes is activated, upregulated, downregulated, or deleted. In certain embodiments, the conversion of a carbohydrate source to a essential amino-acids is under anaerobic conditions. In certain embodiments, the conversion of a carbohydrate source to essential amino acid, alcohol and lipids is under conditions of anaerobic respiration enabled by addition of a terminal electron acceptor other than oxygen (such as a nitrate, nitrite, chlorate, perchlorate, fumarate, or a hypochlorous ion).

In particular aspects of the invention, the amino-acids produced by the recombinant microorganism is in form of free amino acid. In certain aspects, the essential amino-acids are part of some proteins or enzymes that make up the yeast biomass. In some embodiments, the alcohol derivative is either of C₁ to C₂₀. In some other embodiments, the alcohol derivatives are simple alcohols (like Methanol, Ethanol, Propanol, Butanol) or branched alcohols, poly-ols. The lipids that are produced in certain aspects of this invention consist of triglycerides, phospholipids, free fatty acids, fatty-alcohols, fatty-aldehydes, fatty-ester, an unsaturated fatty acid; a branched-chain fatty acid; a branched methoxy fatty acid; a multi-methyl branched acid and combinations of these lipid derivatives.

In certain aspects of the invention, the lipid derivative produced by the recombinant microorganism comprises a carbon backbone of C₄-C₄₀ and combinations thereof. In one embodiment, the lipid derivative comprises a carbon backbone of C₁₀, C₁₂, C₁₄ C₁₆ and C₁₈.

In some aspects of the invention, the carbohydrate source converted to essential amino-acid or protein, lipid and alcohol during the bioprocess is from biomass or from carbohydrates, such as a sugar or a sugar alcohol. In one embodiment, the carbohydrate source converted to a lipid is a lignocellulosic material or from sugarcane or beet molasses. In some cases, the carbohydrate is a monosaccharide (e.g., glucose, fructose, galactose, xylose, arabinose, rhamnose, galacturonic acid, xylitol, sorbitol, or ribose), a disaccharide (e.g., sucrose, cellobiose, maltose, or lactose), an oligosaccharide (e.g., xylooligomers, cellodextrins, or maltodextrins), or a polysaccharide (e.g., xylan, cellulose, starch, mannan, or pectin).

In the particular aspect of the invention, the expression of aspartate aminotransferase is upregulated. In certain embodiments, the PEP-Oxaloacetate transcarboxylase is heterologously expressed by sourcing the DNA sequence from by a polynucleotide from bacterial sources.

In another aspect of the invention, the glycerol 3 phosphate dehydrogenase pathway which deals with glycerol synthesis during anaerobic fermentation process is downregulated so that the NADH used up in the process is diverted to and is made available for biosynthesis of lipids and essential amino-acids during the anaerobic growth phase of the microorganism.

In another aspect of the invention, the fatty acid degradation pathway of the organism is downregulated by the means of downregulating the fatty acid activase enzyme system (FAA)—most notably FAA1, FAA2, FAA3, FAA4 genes. These genes catalyze first step of lipid biodegradation pathway (β-oxidation or Omega Oxidation pathway). As a consequence of this downregulation other genes such as TES1, TES2, ARO8, ARO9, ARO10, CAT2, CAT1 etc are automatically downregulated as a part of above pathway engineering steps. In another aspect of this invention, these genes are downregulated using molecular biology tools.

In some aspects of the invention, one of the engineered metabolic pathways further comprises the conversion of pyruvate and CoA-SH into acetyl-CoA and CO₂ and NAD(P)H.

In some aspects of the invention, one or more of the native enzymes in the engineered metabolic pathways are downregulated or deleted. In certain embodiments, the downregulated or deleted native enzyme is an enzyme involved in central metabolism. In some embodiments, the downregulated or deleted native enzyme is selected from the group consisting of a pyruvate kinase; a hydrogenase; a lactate dehydrogenase; a phosphotransacetylase; an acetate kinase; an acetaldehyde dehydrogenase; an alcohol dehydrogenase; a pyruvate formate lyase; a pyruvate decarboxylase; an enzyme involved in degradation of fatty acids and their derivatives; and combinations of thereof.

In some aspects of the invention, the microorganism is a thermophilic or a mesophilic bacterium.

Another aspect of the invention relates to a process for converting a carbohydrate source to a lipid comprising contacting the carbohydrate source with a recombinant microorganism of the invention. In some embodiments, the carbohydrate source comprises lignocellulosic biomass. In certain embodiments, the lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, agave, and combinations thereof. In other embodiments, the carbohydrate source comprises a carbohydrate. In certain embodiments, the carbohydrate is a sugar, a sugar alcohol, or a mixture thereof.

In some aspects of the invention, the lipid produced by the recombinant microorganism is secreted. Another aspect of the invention relates to an engineered metabolic pathway

for producing a lipid from consolidated bioprocessing media.

One aspect of the invention relates to a recombinant microorganism comprising a native and/or heterologous enzyme that converts oxaloacetate and acetyl-CoA to malonyl-CoA and pyruvate, wherein said one or more native and/or heterologous enzymes is activated, upregulated, downregulated, or deleted. In some embodiments, the microorganism produces a hydrocarbon. In some embodiments, the enzyme is a transcarboxylase. In one embodiment, the transcarboxylase is encoded by a polynucleotide from Thermoanaerobacter species, P. freudenreichii P. acnes, or C. thermocellum. In another embodiment, the transcarboxylase is genetically modified.

In some embodiments, the genetic modification produces an altered catalytic activity and/or an altered substrate specificity to improve the conversion of a substrate to a product as compared to the native enzyme. In some embodiments, the genetic modification alters catalytic activity and/or substrate specificity to provide a genetically modified polypeptide that converts a substrate to a product that is not catalyzed by the native enzyme in vivo, or is catalyzed at only minimal turnover.

### Part 1: Anaerobic Biosynthesis of Essential Amino Acids—Pathway Engineering in a Eukaryotic Host Microorganism

One aspect the invention provides a genetically modified eukaryotic microorganism comprising one or more native and/or heterologous enzymes that function in one or more engineered metabolic pathways to convert a carbohydrate source to an essential amino acids, wherein the one or more native and/or heterologous enzymes is activated, upregulated, downregulated, or deleted. The one or more individual genetic modifications to the host cell are detailed below:

### a) Increased Aspartate Aminotransferase (Enzyme Commission Number 2.6.1.1) Activity to Increase Flux Between Oxaloacetate and Aspartate by Overexpressing Genes AAT2, and Optionally ARO8 in the Genetically Modified Eukaryotic Host Microorganism

Transamination of oxaloacetic acid and alpha-ketoglutaric acid is the standard entry point of metabolic carbon into amino-acid biosynthesis pathway. During aerobic respiration the oxaloacetic acid is produced in the course of Krebs cycle which is then acted upon by Aspartate Aminotransferase enzyme (encoded by AAT2 and AAT1 genes). In one embodiment, the native AAT2 and/or AAT1 genes (e.g. SEQ ID NO's. 13 or 15 encoding SEQ ID NO's. 14 or 16) in the host microorganism are placed under influence of a strong promotor for overexpression (FIG. 2; example 1). In another aspect of this invention the said genes which encode the given enzyme are derived from a heterologous bacterial, fungal or plant source. In this transamination reaction, the amine group is donated to oxaloacetate by glutamine thereby converting it to aspartate/aspartate semialdehyde. The glutamate is produced by transamination of alpha-ketoglutarate. In one aspect of this invention the organism is grown under anaerobic conditions in the presence or absence of a secondary terminal electron acceptor (either throughout the course of batch/fed-batch or for a small part of the batch) like Nitrate, Nitrite, Chlorate, Perchlorate, Fumarate, Hypochlorous ions, for example nitrate.

Once converted to aspartate/aspartate semialdehyde, and under anoxic conditions, the internal metabolic pathways of the microorganism channel aspartate towards lysine or threonine, cysteine, methionine, all of which are essential amino-acids and are important components of any protein rich dietary supplement for mammals. Therefore, expression of the gene (ARO8 e.g. SEQ ID NO. 19) in the host microorganism, which encodes an enzyme named 2-aminoadipate transaminase (e.g. SEQ ID NO. 20) that catalyzes conversion of 2-oxoadipate to 2-aminoadipate, may also be upregulated.

Mammals are incapable of producing essential amino acids and need to get these amino-acids from diet which they consume. Therefore, this change forms the first step towards anaerobic co-production of high protein content biomass to be utilized for the purpose of animal or fish fodder or other applications involving dietary supplements for mammals or production of their derivatives.

b) Heterologous Expression of Phosphoenolpyruvate Carboxykinase (Enzyme Commission Number 4.1.1.32) from Corynebacterium Glutamicum by Replacing a Native PCK1 Gene in the Genetically Modified Eukaryotic Host Microorganism

A second important genetic modification of the host microorganism is overexpression of the native and/or heterologous gene PCK1 (e.g. SEQ ID NO. 17), which encodes phosphoenolpyruvate carboxykinase (e.g. SEQ ID NO. 18). This enzyme is a cytosolic enzyme which mediates biocatalytic conversion of Phosphoenolpyruvate (PEP) to oxaloacetate, via a combination of carboxylation and substrate-level-phosphorylation reactions in single step. The three carbon PEP is carboxylated by this enzyme to produce 4 carbon oxaloacetic acid. In the process the phosphate group from PEP is transferred to GTP by the means of phosphorylation.

In an anoxic or anaerobic process (process devoid of aeration) where oxygen is absent as terminal electron acceptor, the fate of pyruvate differs from the central carbon metabolism seen during aerobic respiration. Instead, the flux moving through the Krebs cycle is minimal and usually in reverse direction, producing intermediates such as oxaloacetate, succinate and alpha-ketoglutarate. PEP-Carboxykinase provides an important entry-point for metabolic carbon into this reductive Krebs cycle necessary for amino-acid biosynthesis. Since in anaerobic fermentation/respiration, the availability of energy is hard-pressed, this GTP-producing reaction is improves the energy balance of cell-cycle as a whole.

In one aspect of this invention, the PEP-carboxykinase is upregulated by means of placing it under the control of a strong promotor (see example 1). In another aspect of invention, the PEP-carboxykinase gene that is cloned into the microbial host cell is one that is derived from a heterologous bacterial, fungal and plant source. The upregulation of PEP Carboxykinase will meet three criteria: firstly, it will increase the cytosolic pool of oxaloacetic acid available for transamination. Secondly it will contribute to energy economics of the cell growing in anoxic conditions by providing a GTP by substrate level phosphorylation. Thirdly, by channelling part of pool towards essential amino acid synthesis, it will increase the protein content of the fermenting organism making it robust for withstanding the pressures of fermentation. The upregulation is depicted in FIG. 2, by presence of a bold arrow indicating increased metabolic flux through the pathway.

### c) Downregulation of Cytosolic Branched-Chain Amino Acid (BCAA) Aminotransferase (Enzyme Commission Number 2.6.1.42) Activity by Deleting BAT2 Gene or Placing Under Influence of Weak Promoter in the Genetically Modified Eukaryotic Host Microorganism

Branched-chain amino acid aminotransferase is involved in catabolism of amino-acids and is responsible for production of various keto-acid analogues which are further reduced to various diols such as iso-amyl alcohol and other fusel-oils. The pathway catalysed by this enzyme, it is a major NADH sink for recycling the NAD in order to keep the glycolysis and fermentative ethanol production pathway operational. In some aspects of this invention, said BAT2 gene (e.g. SEQ ID NO. 23) in the host microorganism is either deleted or downregulated by placing it under the influence of weak promotor. Alternatively the BAT2 gene is deleted while BAT1 is placed under the control of a weak promotor (see FIG. 2).

### d) Downregulation of Threonine Aldolase (Enzyme Commission Number 4.1.2.48) Activity by Deleting or Placing GLY1 Gene Under Influence of Weak Promoter in the Genetically Modified Eukaryotic Host Microorganism

Threonine Aldolase is an enzyme involved in catabolism of essential amino-acid threonine. It catalyzes lysis of threonine and eventual production of acetaldehyde.Degradation of threonine produced by the host cell microorganism can be prevented by down-regulating expression of the gene GLY1 (e.g. SEQ ID NO. 25) which encodes for this enzyme (e.g. SEQ ID NO. 26), by either deleting the GLY1 gene or placing it under the control of a weak promotor (see example 1).

e) Overexpression of a Gene Cassette of Homoaconitase (Aco2p) (Enzyme Commission Number 4.2.1.36), Homocitrate Synthase (Enzyme Commission Number: 2.3.3.1) and Homoisocitrate Dehydrogenase (Enzyme Commision Number: 1.1.1.87) in the Genetically Modified Eukaryotic Host Microorganism

The Aco2P protein (e.g. SEQ ID NO. 140 encoded by a LYS4 gene such as SEQ ID NO. 139) plays an important role in isomerizing homocitrate molecule to homoisocitrate. Homoisocitrate is then oxidized to alpha keto adipic acid by means of homoisocitrate dehydrogenase (e.g. SEQ ID NO. 144 encoded by a LYS12 gene such as SEQ ID NO. 143). The alpha ketoadipic acid is then transaminated by a nitrogen donor and an electron carrier to alpha-aminoadipic acid which then enters the lysine biosynthesis pathway for lysine overproduction. The homocitrate synthase(s) (e.g. SEQ ID NO. 142 encoded by a LYS20 gene such as SEQ ID NO. 141; SEQ ID NO. 164 encoded by a LYS21 gene such as SEQ ID NO. 163) and homoisocitrate dehydrogenase (SEQ ID NO. 144 encoded by a LYS12 gene such as SEQ ID NO. 143) act in syncrony with TCA cycle genes cloned in the cytosol of fermenting organism to channel the metabolic flux from TCA cycle towards essential amino acid (lysine) biosynthesis pathway via alpha aminoadipate step.

### f) Expression of Bacterial Krebs Cycle Genes in Cassette in Cytosol: Citrate Synthase (Enzyme Commission Number 2.3.3.16), Isocitrate Dehydrogenase (1.1.1.42) in the Genetically Modified Host Microorganism

The Krebs (Tricarboxylic acid (TCA)) cycle is an important biochemical pathway in central carbon metabolism. It plays an important role not only in generating reduction equivalent (NADH) which is necessary for both anabolic processes and energy generation in electron transport chain. The current invention deals with anaerobic production of the essential amino acids, lipids and alcohols. All these three biochemical reactions are reducing anabolic reactions. In anaerobic conditions, the electron transport system is not operational due to the absence of terminal electron acceptor, oxygen. However, there are various candidate molecules which perform the role of terminal electron acceptors instead of oxygen. They include nitrates, nitrites, chlorates, perchlorates, fumarate or hypochlorous ions. Furthermore, the enzymes performing the catalysis of biochemical reactions of TCA cycle are localized in mitochondria. In order to generate sufficient reducing power, in the form of NADH, required to drive the anabolic pathways leading to amino acid, lipids and alcohol biosynthesis the first two enzymes of the TCA cycle, namely citrate synthase and isocitrate dehydrogenase, are expressed in cytosol of the host microorganism. A suitable cytosolic citrate synthase is for example SEQ ID NO. 154 encoded by a CIT gene such as SEQ ID NO. 153.)

### Part 2: Anaerobic Biosynthesis of Lipids by Pathway Engineering in a Host Microorganism

A further aspect of the invention provides a genetically modified eukaryotic microorganism comprising one or more native and/or heterologous enzymes that function in one or more engineered metabolic pathways to convert a carbohydrate source into both essential amino acids and lipids, wherein the one or more native and/or heterologous enzymes is activated, upregulated, downregulated, or deleted. The engineered metabolic pathways of the host microorganism engineered for anaerobic co-production of lipids, as shown in FIG. 3. A terminal electron acceptor in form of nitrate, fumarate, chlorate, perchlorate, hypochlorous ion, may be added to the fermentation medium during cultivation of the host microorganism. The individual genetic modifications to the host cell are detailed below:

a) Replacement of Native Cytosolic Fatty Acid Synthase Complex (FAS2) of the Genetically Modified Eukaryotic Host Microorganism with Heterologously Expressed Bacterial Genes acpS (Enzyme Commission Number 2.7.8.7), acpP, fabB (Enzyme Commission Number 2.3.1.41), fabD (Enzyme Commission Number 2.3.1.39), fabG (Enzyme Commission Number 1.1.1.100), fabH (Enzyme Commission Number 2.3.1.41), fabI (Enzyme Commission Number 1.3.1.39), fabZ (Enzyme Commission Number 4.2.1.59)

Fatty acid synthesis is essentially a 4-step cyclical process wherein two carbon-atoms are linked and reduced sequentially to produce a saturated alkane backbone of the fatty acid.

The fatty acid synthesis in eukaryotic microorganisms (e.g. yeast) is catalysed by a multi-enzyme complex known as FAS-2 which is located in cytosol. In view of the complexity of this single unit enzyme, it is not easily amenable to engineering of the individual reactions of the fatty acid biosynthesis pathway. Bacterial fatty acid synthesis, on the other hand, is composed of individual enzymes that catalyse fatty acid biosynthesis pathway in the cytoplasm and are encoded by separate genes controlled by separate promotors that are easier to manipulate.

Accordingly, in a preferred embodiment, the gene(s) encoding the entire fatty-acid synthase complex in the host microorganism (FAS2 gene encoding the fatty acid synthase complex enzyme; (e.g SEQ ID NO: 31; encoding SEQ ID NO: 32) were deleted and replaced with a codon-optimized cassette of individual bacterial genes, and operably linked to a eukaryotic promoter, that encode enzymes responsible for catalyzing fatty acid biosynthesis pathway native to bacteria. This ensures localization of biosynthesis in cytosol of the fermenting organism thereby making it easier for secretion and storage etc. In one aspect of this invention, the fatty acid synthesis genes are sourced from an obligatory anaerobic bacterium that is a native of an anaerobic environment. A suitable gene cassette encoding bacterial fatty acid synthase pathway enzymes are exemplified in example 1 (comprising the genes acpS (e.g SEQ ID NO: 33; encoding SEQ ID NO: 34); acpP (e.g SEQ ID NO: 35; encoding SEQ ID NO:36), fabB (e.g SEQ ID NO: 37; encoding SEQ ID NO:38), fabD (e.g SEQ ID NO: 39; encoding SEQ ID NO:40), fabG (e.g SEQ ID NO: 41; encoding SEQ ID NO:42), fabH (e.g SEQ ID NO: 43; encoding SEQ ID NO:44), fabl (e.g SEQ ID NO: 45; encoding SEQ ID NO:46), and fabZ (e.g SEQ ID NO: 47; encoding SEQ ID NO:48).

### b) Heterologous Expression of Codon Optimized Acyl-ACP Thioesterase (Enzyme Commission Number 3.1.2.14) for Production of Short and Medium Chain Fatty Acids in the Genetically Modified Eukaryotic Host Microorganism

Acyl-Acyl Carrier Protein Thioesterase catalyzes the cleavage of fatty acid chains growing in the iterative fatty acid synthesis pathway from acyl-carrier protein and subsequently from coenzyme A, thereby releasing a free fatty-acid in the cytosol after it reaches a certain chain-length. The chain-length of 16 and 18 carbons is predominantly seen in most life forms. For further elongation of the fatty acid chain, there are other enzyme systems employed by various living organisms.

Few organisms are known to produce shorter (or longer) chain-lengths of fatty acids and have evolved special variants of this thioesterase enzyme to produce such specific chain-lengths. According to the present invention, the native acyl-acp thioesterase of the fermenting organism is replaced with a codon-optimized thioesterase derivable from organisms known to produce shorter and medium chain fatty acids. Various plants like coconut, cuphea etc. are known to produce C12 (Lauric acid) predominantly among its pool. In one aspect of this invention, the thioesterase from such sources (for example the Cuphea wrightii gene (e.g. SEQ ID NO 117 encoding an acyl-acp thioesterase SEQ ID NO 118) is cloned in the genome of microorganism to ensure prevalence of shorter and medium chain fatty acids in the lipid profile secreted by the microorganism. As seen in FIG. 11, the effect of this heterologous expression is seen in changed lipid profile (Free fatty acids+triglycerides) produced by the organism. Due to effect of this modification, the yeast starts producing higher proportion of C12 and C14 (Lauric acid and myristic acid respectively).

c) Downregulation of Genes FAA1, FAA4, FAA3 and FAA2 to Reduce with Fatty Acyl-CoA Synthetase Activity (Enzyme Commission Number 6.2.1.3) in the Genetically Modified Eukaryotic Host Microorganism

Fatty acid activation is the first step in fatty acid catabolism. The reaction is catalyzed by an enzyme known as Fatty Acid Activase which converts free fatty acid to Fatty acyl CoA. This step activates the fatty acid to beta oxidation pathway. This enzyme comes in four paralogs namely FAA1, FAA3 and FAA4 catalyse activation of long chain fatty acids, while FAA2 catalysese the activation of medium chain fatty acids. According to the present invention, these genes (e,g, FAA1 [SEQ ID NO 55]; FAA2 [SEQ ID NO 53; FAA3 [SEQ ID NO 57]; and FAA4 [SEQ ID NO 59]) which encode for enzymes (e.g. FAA1 [SEQ ID NO 56]; FAA2 [SEQ ID NO 54; FAA3 [SEQ ID NO 58]; and FAA4 [SEQ ID NO 60]) catalyzing the first step of lipid catabolism are downregulated. FAA2 and FAA1 are deleted whereas others are placed under influence of a weak promotor. The downregulation of FAA genes (notably FAA2) has a positive feedback effect on essential amino-acid catabolism wherein certain genes like ARO9, AR010 etc. are downregulated as a result of FAA downregulation in this strategy. So these modifications feed in to other pathways thereby having a synergistic effect leading to increased synthesis of essential amino acids and lipids when compared to a wild-type patent host microorganism.

d) Enhanced Acetyl CoA Carboxylase (Enzyme Commission Number 6.4.1.2) Expression Conferred by a Recombinant Endogenous ACC1 Gene Wherein said Gene is Operably Linked to a Heterologous Promoter in the Genetically Modified Eukaryotic Host Microorganism

Acetyl CoA Carboxylase is a cytosolic enzyme which catalyzes the first step of lipid biosynthesis by carboxylating cytosolic acetyl coA and producing Malonyl CoA. Upon conversion into Malonyl CoA, the carbon has to move into the fatty acid biosynthesis pathway. In this invention, acetyl coA carboxylase enzyme is upregulated by placing ACC1 gene (e.g. ACC1 [SEQ ID NO ] which encodes the ACC1 enzyme [SEQ ID NO]) under the influence of a strong promotor (e.g. TEF 1 promoter [SEQ ID NO 5]).

### e) Heterologous Expression of Bacterial Pyruvate Formate Lyase (Enzyme Commission Number 2.3.1.54) to Improve Cytosolic Levels of Acetyl CoA Under Anaerobic Conditions in the Genetically Modified Eukaryotic Host Microorganism

The emphasis of this invention is anaerobic coproduction of essential amino acids and alcohol with side-stream of lipids. Most of the anabolic biosynthesis pathways occur in the cytosol of the organism whereas the catabolic processes happen in the mitochondria or the peroxisomes of the eukaryotic organism. The metabolite Acetyl CoA is an essential part of central carbon metabolism and is predominantly produced in mitochondria during aerobic respiration of the organism by Pyruvate dehydrogenase (PDH) enzyme complex localized in the mitochondria of the organism. During anaerobic or anoxic growth, the PDH is inactive therefore the organism converts pyruvate (coming from glycolysis super fermentation pathway) into acetaldehyde which is subsequently converted to alcohol (ethanol). This bypass is catabolized by another enzyme complex known as Pyruvate decarboxylase (PDC). This enzyme bypasses acetyl CoA and produces acetaldehyde, thus negatively affecting the cytosolic pools of acetyl CoA.

Therefore, another heterologous bacterial system named Pyruvate Formate Lyase genes (e.g. PFLA gene [SEQ ID NO 49] and PFLB gen [ SEQ ID NO 51]which encode respective enzymes [SEQ ID NO 50 & 52])) are cloned into the genetically modified fermenting organism to convert pyruvate to Acetyl CoA and Formic acid. This step ensures there is a steady pool of Acetyl CoA present within the cytosol for the subsequent action by ACC1 and fatty acid synthesis machinery.

f) Overexpression of Formate dehydrogenase (Enzyme Commission Number 1.17.1.9) to Produce NADH/14+ and Metabolize Formic Acid to CO2 and H2O in the Genetically Modified Eukaryotic Host Microorganism

The expression of PFL system results in the production of formate in the cytosol, that can reach levels that are toxic for the normal functioning and growth of the host cells. Therefore, expression of a gene encoding formate dehydrogenase (FDH) in the host microorganism may advantageously upregulated to oxidize formate to carbon dioxide and generation of a redox potential NADH/H⁺ in the process (e.g. upregulating expression of the FDH gene [SEQ ID NO 95 encoding FDH enzyme [SEQ ID NO 96]) with a strong promoter (e.g. TEF1 [SEQ ID NO 5]).

### g) Overexpression of NADH Kinase (Enzyme Commission Number 2.7.1.86) in the Genetically Modified Eukaryotic Host Microorganism

NADH Kinase is an enzyme that catalyzes the transfer of phosphoryl group from Adenosine Triphosphate (ATP) to NADH to yield NADPH. Most of the anabolic biochemical reactions prefer to utilize the required reducing power in form of NADPH. However, most of the reducing power produced by a genetically modified host microorganism obtained from the modifications in 1.c through 1.f. is in the form of NADH. Hence, overexpression of NADH kinase in such host microorganisms can provide the required NADPH by phosphorylatings the NADH produced in the cytosol. The titres of NADH Kinase are increased in the cytosol of the host microorganism by placing the gene encoding the native enzyme (e.g. NADPH kinase gene [SEQ ID NO 161] encoding NADPH kinase enzyme [ SEQ ID NO 162] under the influence of a strong constitutive promotor (e.g. pADH promoter [SEQ ID NO 4]). This maintains the required levels of phosphorylated and dephosphorylated NAD⁺ molecules to be utilized by the bioreactions as needed.

### Part 3: Anaerobic Coproduction of Alcohols by Pathway Engineering in a Host Microorganism

The primary metabolite produced in this process is alcohol (ethanol, butanol etc.) by the means of the ethanol fermentation super-pathway present in the native fermenting microorganism, yeast. The metabolic flux running through this pathway is naturally very high along with the ethanol tolerance of the organism.

a) Downregulation of Glycerol-3-Phosphate Dehydrogenase (Enzyme Commission Number 1.1.1.8) Activity to Pool the NADH Required for other Anabolic Pathways by Downregulation of GPD1 and GPD2 Genes in the Genetically Modified Eukaryotic Host Microorganism

Glycerol synthesis during alcoholic fermentation is the organism's way to restore the redox potential of the fermenting organism by recycling the NADH back to NAD so that the glycolysis continues. In aerobic conditions, NADH is oxidized by a terminal electron acceptor (oxygen) at the end of electron transport chain, so NADH recycling occurs naturally. But in the absence of oxygen as terminal electron acceptor, the organism has to deposit the electrons and the protons it has produced to an acceptor so that the NAD pool suffices for catering to all cellular reactions. This role is naturally served by glycerol which utilizes NADH in its synthesis reaction when GPDH enzyme reduces Dihydroxyacetone phosphate to 3 phospho glycerol.

Genetically modified eukaryotichost microorganisms of the present invention already have many NADH and electron sinks due to the modified essential amino-acid metabolic pathway and lipid metabolic pathways. For this reason, expression of gene(s) (e.g. GPD2 gene [SEQ ID NO 29] encoding of the GPDH enzyme [e.g. GPD2 enzyme [ SEQ ID NO 30] can be down-regulated in order to decrease the production of glycerol in the process. The NADH saved in this reaction can then be utilized for NADPH-intensive reactions that catalyse the amino-acid and lipid biosynthesis pathway in the host microorganism.

### b) Downregulation of Alcohol Dehydrogenase Enzyme Activity (Enzyme Commission Number: 1.1.9.1) in the Genetically Modified Eukaryotic Host Microorganism

There are multiple alcohol dehydrogenases which specialize in multiple anabolic and catabolic biochemical reactions pertaining to alcohol biosynthesis pathway in fermenting organisms. The Ethanol biosynthesis pathway in fermenting yeast is a super-pathway which a very strong metabolic flux moving through the pathway. In order to reduce the strength of this metabolic flux, the two out of 6 Alcohol Dehydrogenase (ADH) genes, namely ADH1 gene [e.g. SEQ ID NO 129] and ADHS gene [e.g. SEQ ID NO 135] can be deleted or placed under the control of a weak promoter. This will slightly reduce the velocity of flux flowing through this pathway thereby making it easy for the other interventions in Part 1 and Part 2 of this invention to act and divert the flux towards other metabolic pathways of interest as described in part 1 and part 2 of this document.

The sequence listing that forms part of the present application discloses the sequence of each of the genes and their encoded proteins described above; and includes a number of homologous proteins and their encoding genes suitable for implementing the present invention.

### Part 4: Role of Alternate Terminal Electron Acceptor for the Genetically Modified Eukaryotic Host Microorganism

The role of an alternate “terminal electron acceptor” is an important feature of this invention. Under aerobic growth, oxygen acts as the electron acceptor. This creates a gradient along the electron transport chain in mitochondria which results in generation of energy. In the absence of oxygen, ethanol acts as a terminal electron acceptor (or an electron sink where NADH⁺ can deposit its H⁺ and e⁻ to be recycled to keep glycolysis going). Ethanol is not as strong a terminal electron acceptor as oxygen; and only generates 2 net ATP per glucose. An alternate terminal electron acceptor is a compound with the reducting potential that quantitatively lies between oxygen and ethanol. It is not as efficient as oxygen (which can theoretically yield 36 ATP per glucose) but is stronger than ethanol thereby giving a “respiratory assist” to the fermenting organism to produce more energy and reducing power than it does in usual anaerobic process. In addition to being a life-support, some candidates for performing this task (like nitrates) are also nitrogen donors and play a role in amino-acid metabolic pathways. In a process which aims at producing amino acids as the coproduct, it is important to improve the C/N ratio of fermentation process. The “nitrogen donor” part of this is helpful (but no essential) to the amino acid biosynthesis. The “respiratory assist” part is important to both amino acid and lipid biosynthesis part of this process (and by corollary detrimental to ethanol biosynthesis pathway). The use of alternate terminal electron acceptor as a respiratory assist in combination with targeted pathway modification is an important feature of this invention. Because then, it can be used for any strategy to produce any compound. It is important to note that addition of an alternate terminal electron acceptor is not a mandatory. Providing an alternate terminal electron acceptor improves the flux through the designed pathway but not providing alternate terminal electron acceptor does not stop the required fluxes from flowing through the designed pathway. Suitable terminal electron acceptors for performing this biochemical function imay be selected from among: a nitrate, a nitrite, a fumarate, a chlorate, a perchlorate or a hypochlorous acid ion. Additionally, the method of fermentation may include alternate cycles of anaerobic respiration and fermentation to enhance co-production of amino-acids, alcohol and lipids.

### Part 5: Host Cell for Production of Essential Amino Acids and Optionally the Co-Production of One or More Co-Products

The genetically modified eukaryotic microorganism of the invention can be engineered in a suitable parent host cell. In one embodiment, the host cell is a yeast cell for example, a species belonging to a genus selected from the group consisting of Saccharomyces, Kluyveromyces, Pachysolen, Candida, Pichia, Schizosaccharomyces, Hansenula, Kloeckera, Schwanniomyces, Rhodutorula and Yarrowia. Yeast species as host cells may include, as an example, S. cerevisiae, S. bulderi, S. barnetti, S. exiguus, S. uvarum, S. diastaticus, K. lactis, K. marxianus, or K. fragilis. In some embodiments, the yeast is chosen from the group consisting of yeast, Schizzosaccharomyces pornbe, candida, Pichia pastoris, Pichia stipitis, Yarrowia lipolytica, Pachysolen tannophilus, Rhodotorula gracilis, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is baker's yeast. In another embodiment, the yeast may be a thermotolerant yeast or yeast capable of secreting thermotolerant enzymes like phospholipases. The host cells can contain antibiotic markers or can contain no antibiotic markers.

### EXAMPLES

### Materials and Methods

### Construction of Genetically Engineered Microbial Strains

Saccharomyces cerevisiae wild type strain was used as a host for engineering all the reported genetic modifications. All the strains were maintained in 25% glycerol in frozen vials at −80° C. The wild type yeast strain (known as WT henceforth) was cultivated in YPD medium (10 g/l yeast extract, 20 g/l Bacto Peptone and 20 g/L glucose). Selection of yeast transformants with either a variety of selection markers was performed by growth on a yeast minimal medium (6.7 g/l of Yeast Nitrogen Base (Difco), 20 g/l glucose, and a mixture of appropriately selected marker related additions (i.e. antibiotic selection etc). Yeast cells were cultivated at 30° C. in closed Erlenmeyer flasks and shaken at 150 rpm. The competent E.coli cells from DH5α-cell line was purchased from Thermo-Fisher and were utilized for plasmid preparation. E. coli was grown in Lysogeny broth (LB) medium at 37° C. Ampicillin at 100 μg/ml was added to the medium when required.

Gene modifications were generated using a previously reported gene disruption based on homologous recombination in S. cerevisiae (Baudin, Ozier-Kalogeropoulos et al. 1993). Gene disruption cassettes containing the selectable markers from variety of sources were produced with homology on either side of each target integration site. Yeast cells were transformed using the Li/Ac/PEG method as previously described by Gietz et al. (Gietz, Jean et al. 1992, Gietz, Schiestl 2007). Following yeast transformations, colonies were selected on the relevant selective media. To verify the genetic stability of the engineered strains, their genomic DNA was isolated (Qiagen Genomic DNA extraction kit) and then subjected to a diagnostic PCR amplification that amplified regions both upstream and downstream of the deletion sites. PCR products were purified (Qiagen PCR Purification kit) and then analysed for the band-size using gel-electrophoresis.

### Genotype of Genetically Modified Yeast Strains of the Invention

| | | | ARO8 | FINAL |
|---|---|---|---|---|
| Genetic feature | Functional feature | SEQ ID NO | STRAIN | STRAIN |
| Δfaa2 | Fatty acid Activase 2 | 53 (gene), | ✓ | ✓ |
| | | 54 (encoded | | |
| | | protein | | |
| | | product) | | |
| Δfaa3 | Fatty acid Activase 3 | 57 (gene) | ✓ | ✓ |
| | | 58 (encoded | | |
| | | protein | | |
| | | product) | | |
| Δfaa1 | Fatty acid Activase 1 | 55 (gene), | ✓ | ✓ |
| | | 56 (encoded | | |
| | | protein | | |
| | | product) | | |
| Δfaa4 | Fatty acid Activase 4 | 59 (gene), | — | 1 |
| | | 60 (encoded | | |
| | | protein | | |
| | | product) | | |
| PACC1::PTEF1 | Promoter pTEF1 replacing | 5 (promoter) | ✓ | ✓ |
| | native promoter to regulate | 79 (gene) | | |
| | ACC1 gene for acetyl CoA | 80 (encoded | | |
| | carboxylase | protein | | |
| ADH5::PFLA (along | Pyruvate Formate Lyase A | 49 (gene) | ✓ | ✓ |
| with PFLB in the | | 50 (encoded | | |
| cassette) | | protein | | |
| | | product) | | |
| ADH5::PFLB (along | Pyruvate formate lyase B | 51 (gene), | ✓ | ✓ |
| with PFLA in the | | 52 (encoded | | |
| cassette) | | protein | | |
| | | product) | | |
| GPD2::Bacterial FAS | Bacterial FAS (fatty acid | 29 (1-460) | ✓ | ✓ |
| Cassette (acpP and | synthase) Cassette regulated | promoter | | |
| acpS and fabB, D, G, | by yeast GPD2 promoter and | 35 (acpP gene) | | |
| H, I, and Z) | Shine Dalgano sequence | 33 (acpS gene) | | |
| | | 37 (fabB gene) | | |
| | | 39 (fabD gene) | | |
| | | 41 (fabG gene) | | |
| | | 43 (fabH gene) | | |
| | | 45 (fabI gene) | | |
| | | 47 (fab Z | | |
| | | gene) | | |
| | | encoding | | |
| | | proteins 36, | | |
| | | 34, 38, 40, 42, | | |
| | | 44, 46, and 48 | | |
| | | respectively. | | |
| Yeast | Cuphea wrightii Thioesterase | 117 (gene), | ✓ | ✓ |
| Thioesterase::Cuphea | (SEQ ID NO 117) replacing | 118 (encoded | | |
| wrightii Thioesterase | native Yeast Thioesterase | protein | | |
| | | product) | | |
| PNADHKinase::PADH | Promoter pADH replacing | 4 (promoter) | ✓ | ✓ |
| | native promoter to regulate | 161 (gene) | | |
| | NADH Kinase gene | 162 (encoded | | |
| | | protein) | | |
| ADH1::Bacterial | Bacterial Citrate Synthase | 153 & 155 | ✓ | ✓ |
| Krebs Cycle genes | and Isocitrate dehydrogenase | (genes), | | |
| bacterial Citrate | expressed from a cassette | 154 & 156 | | |
| Synthase, Isocitrate | | (encoded | | |
| Dehydrogenase | | protein | | |
| expressed in a cassette | | products) | | |
| for their cytosolic | | | | |
| expression | | | | |
| ΔGPDH1 | Glycerol 3 phosphate | 29 (gene), | ✓ | ✓ |
| | Dehydrogenase GPD2 | 30 (encoded | | |
| | | protein | | |
| | | product) | | |
| ΔADH1 | Alcohol dehydrogenase 1 | 129 (gene), | ✓ | ✓ |
| | | 130 (encoded | | |
| | | protein | | |
| | | product) | | |
| ΔADH5 | Alcohol dehydrogenase 5 | 135 (gene), | ✓ | ✓ |
| | | 136 (encoded | | |
| | | protein | | |
| | | product) | | |
| ΔGLY1 | Threonine aldolase | 25 (gene), | ✓ | ✓ |
| | | 26 (encoded | | |
| | | protein | | |
| | | product) | | |
| PAAT1::PGPD1 | Promoter pGPD1 replacing | 1 (promoter) | ✓ | ✓ |
| | native promoter to regulate | 15 (nt 1-520 | | |
| | AAT1 gene for aspartate | Δ) | | |
| | aminotransferase | 15 (nt 521- | | |
| | | 1876: gene) | | |
| | | (encoded | | |
| | | protein | | |
| | | product) | | |
| YeastPCK1::bacterial | Bacterial PCK1 replacing | 17 (nt 1-460 | ✓ | ✓ |
| PCK1 | native Yeast PCK1 for PEP | promoter) | | |
| | carboxykinase | 17 (nt 461- | | |
| | | 2110 Δ gene) | | |
| | | 137 (gene) | | |
| | | 138 (encoded | | |
| | | protein | | |
| | | product) | | |
| PLYS20::PCYC1 | Promoter pCYC1 replacing | 3 (promoter) | ✓ | ✓ |
| | native promoter to regulate | 141 (gene) | | |
| | LYS20 gene for homocitrate | 142 (encoded | | |
| | synthase | protein | | |
| | | product) | | |
| PLYS4::PADH1 | Promoter pADH1 (SEQ ID | 4 | ✓ | ✓ |
| | NO 4) replacing native | | | |
| | promoter to regulate LYS4 | | | |
| | gene for Homoaconitase | | | |
| FAA2::Lys20 | Homocitrate Synthase | 141 (gene), | — | ✓ |
| | | 142 (encoded | | |
| | | protein | | |
| | | product) | | |
| PLYS12::PZEV1-2 | Promoter pZEV1-2 replacing | 2 (promoter) | ✓ | ✓ |
| | native promoter to regulate | 143 (gene) | | |
| | LYS12 gene for | 144 (encoded | | |
| | Homoisocitrate | protein | | |
| | dehydrogenase | product) | | |
| PLYS21::PPGK1 | Promoter pPGK1 replacing | 7 (promoter) | — | ✓ |
| | native promoter to regulate | 163 (gene) | | |
| | LYS21 gene for homocitrate | 164 (encoded | | |
| | synthase | protein | | |
| | | product) | | |
| FAA1::Cgl0248 | Homocitrate Synthase | 145 (gene), | ✓ | ✓ |
| | | 146 (encoded | | |
| | | protein | | |
| | | product) | | |
| FAA3::PP3:ARO8 | Aromatic Transaminase for | 9 (promoter) | ✓ | — |
| (FAA3 locus replaced | conversion of Ketoadipic | 19 (nt 461- | | |
| with heterologous | acid to Amino adipic acid | 1960: gene) | | |
| ARO8 under influence | | 20 (encoded | | |
| of P3 promoter for | | protein | | |
| overexpression) | | product) | | |
| ΔBAT2 | Cytosolic Branched chain | 23 (gene) | ✓ | — |
| | Amino acid | 24 (encoded | | |
| | Aminotransferase | protein | | |
| | | product) | | |

“✓” indicates that the genome of the strain comprises the respective genetic modification; while
“—”indicates that the genome of the strain lacks the respective genetic modification

### Batch Fermentations

Each yeast strain was cultivated in conical shake flasks, in a culture volume of 1000 ml. All cultivations were performed at 30° C. Duplicates of all batches were performed; results shown are mean values (and standard error) for two identical processes. The fermentation experiments lasted for 60 hours in anaerobic shaker-incubator. Samples (˜10ml) were taken every 4 hours under aseptic conditions and used OR stored for analytical purposes. 1 ml of the sample was used to calculate dry cell weight (DCW), 1 ml for HPLC data and 10 ml for liquid-liquid extraction and lipid quantification.

### Analytical Methods

Yeast cell growth was monitored by measuring OD600 nm with a Shimadzu UV mini-1240 spectrophotometer (Shimadzu, Japan). Samples were taken periodically from the fermenters, filtered through a 0.22 μm syringe filter and supernatants were preserved at −20° C. for later HPLC analysis and liquid-liquid extraction. Cell dry weight (CDW) was measured by centrifuging 1 ml of culture at 8000 g for 20 min in a pre-weighed Eppendorf tube. The cells were washed twice with distilled water and then were dried in a 70° C. oven until a constant weight. Cell dry weight was correlated with OD600 using a standard curve (1 OD600=0.38 grams/litre). An Ultimate 3000 high-pressure liquid chromatography system (Dionex, Sunnyvale, CA) equipped with an Aminex HPX-87H column (Bio-Rad, Hercules, CA) and a Shodex RI-101 detector (Showa Denko KK, Tokyo, Japan). The column oven temperature was set to 30° C., and the mobile phase consisted of 5 mM H2SO4 with a flow rate of 0.3 ml/min.

Intracellular lysine content of the yeast cells was determined by collecting the yeast cells by centrifugation and washing them twice with sterilized water. The harvested and washed cells were re-suspended in sterilized water, and the suspension was adjusted to an OD600 of 20. Subsequently, the intracellular amino acids in an aliquot (0.5 ml) of the cell suspension were extracted by boiling in water at 100° C. for 20 min. After centrifugation, each supernatant was subsequently quantified with an amino acid analyzer by ion-exchange chromatography and post-column ninhydrin derivatization (JLC-500/V2; JEOL). The content of each amino acid was represented as g per g dry cell weight (DCW).

### Liquid-Liquid extraction of lipids

The 10 ml sample was subjected to downstream processing for the extraction of fatty acids and lipids in the medium. The cells were removed from the liquid phase by the means of centrifugation of the medium at 5000 rpm for 6 minutes. The methods of intracellular and extra-cellular fatty acid extraction are as described by Cocito and Delfini (Cocito, Delfini 1994) using diethyl ether as organic solvent. The solvent was evaporated by the means of nitrogen gas at 35° C. The residual lipids is weighed (by subtracting the pre-weighed falcon-tubes) and approximate volume measured (by means of micropipette). In case of storage, the samples were stored at the temperature of −20° C. in freezer.

Free fatty acid (FFA) and triglyceride (TG) Assay

The levels of free fatty acids and neutral lipids (mono/di/Triglycerides) were measured in the plasma using the BioVision free fatty acid colorimetric quantification kit (http://www.biovision.com/free-fatty-acid-quantification-colorimetric-fluorometric-kit-2908.html) and BioVision TG colorimetric quantification kit (http://www.biovision. com/triglyceride -quantification-colorimetric-fluorometric-kit-2917 .html) according to the manufacturer's recommendation. One μl of sample was measured against a standard of varying concentrations of palmitic acid (provided by the kit) and the O.D. was measured at 570 nm in a 96-well Tecan microplate reader. For TG quantification, the samples were incubated for 5 min at 37° C. and measured on a Tecan microplate reader at 600 nm absorbance. Quantification was based on a standard curve derived by linear dilution of the standards included in the respective kits. The levels of both free fatty acid and triglyceride samples were calculated using the slope of the standard curve and the yields obtained were converted to the scale of grams/liter and reported.

Gas Chromatography—Mass Spectrometry conditions for analysis of free fatty acids and triglycerides:

The final lipid analysis for chain-length was done using gas-chromatography mass spectrometry (GCMS). The column used is DBI capillary column, 30 m long and 0.25 mm i.d; film thickness 0.25 1.1m. Initial temperature 60° C. for 2 min. Ramp to 280° C., hold 6 min, Inj=270° C., volume=0 μl, split =30:1, carrier gas He, Solvent delay=2.50 min, Transfer Temp=200° C., Source Temp=150° C., Scan :33 to 450 Da, Column 30.0 m×250 μm.

### Results

As seen in FIGS. 5 to 11, the genetically modified yeast strains of the invention channel carbon metabolism towards the synthesis of free amino acids (as measured by lysine levels), as well as lipids (free fatty acids and neutral lipids (mono/di/triglycerides)) during anaerobic fermentation, such that the amounts of these metabolic products produced during fermentation are increased several fold when compared to the amounts of these products produced by a corresponding parent wild-type yeast strain (from which the genetically modified strains were derived) when cultured under similar conditions. The re-direction of carbon flow in the genetically modified yeast strains leads to lower levels of biomass and ethanol production as compared to the parent wild type yeast strain. The modified strains produced higher proportion of C12 fatty acid (and its analogous triglycerides) as compared to parent wild-type stain.

### Embodiments of the invention

Embodiment 1. A genetically modified microorganism comprising one or more engineered metabolic pathways to simultaneously and anaerobically convert a carbohydrate source (like starch, molasses and lignocellulose) into protein rich “Dried Distillers Grains with Solubles” (DDGS) and/or high protein concentrate enriched with essential amino acids, ethanol and lipids, wherein the said microorganism characterised by:Increased Aspartate Aminotransferase (Enzyme Commission Number 2.6.1.1) pathway to increase flux between Oxaloacetate and Aspartate by overexpressing genes AAT2 and AAT1, Heterologous expression of phosphoenolpyruvate carboxykinase (Enzyme commission Number 4.1.1.32) activity by replacing and overexpressing native PCK1 gene, Upregulation of 2-aminoadipate aminotransferase (Enzyme commission number 2.6.1.39) which is encoded by gene ARO8, Downregulation of Phenylpyruvate decarboxylase (Enzyme Commission Number E.C.4.1.1.43) which is encoded by AR010 under weak promoter. Flux through Ehrlich Pathway is downregulated, Downregulation of Cytosolic branched-chain amino acid (BCAA) aminotransferase (Enzyme Commission Number 2.6.1.42) pathway by deleting or placing BAT2 gene under influence of weak promoter. Downregulation of Threonine Aldolase (Enzyme Commission number 4.1.2.48) pathway by deleting or placing GLY1 gene under influence of weak promoter.

Embodiment 2. A genetically modified microorganism comprising one or more engineered metabolic pathways to simultaneously and anaerobically convert a carbohydrate source (like starch, molasses and lignocellulose) into protein rich “Dried Distillers Grains with Solubles” (DDGS) and/or high protein concentrate enriched with essential amino acids, ethanol and lipids, wherein the said microorganism characterised by Downregulation of glycerol-3-phosphate dehydrogenase (Enzyme Commission Number 1.1.1.8) activity to pool the NADH required for other anabolic pathways by downregulation of GPD1 and GPD2 genes.

Embodiment 3. A genetically modified microorganism comprising one or more engineered metabolic pathways to simultaneously and anaerobically convert a carbohydrate source (like starch, molasses and lignocellulose) into protein rich “Dried Distillers Grains with Solubles” (DDGS) and/or high protein concentrate enriched with essential amino acids, ethanol and lipids, wherein the said microorganism characterised by: Replacement of native cytosolic Fatty Acid Synthase complex (FAS2) of yeast with heterologously expressed bacterial genes acpS (Enzyme Commission Number 2.7.8.7), acpP, fabB (Enzyme Commission Number 2.3.1.41), fabD (Enzyme Commission Number 2.3.1.39), fabG (Enzyme Commission Number 1.1.1.100), fabH (Enzyme Commission Number 2.3.1.41), fabl (Enzyme Commission Number 1.3.1.39), fabZ (Enzyme Commission Number 4.2.1.59), Heterologous expression of codon optimised Acyl-ACP thioesterase (Enzyme commission number 3.1.2.14) for production of short and medium chain fatty acids; Downregulation of genes FAA1, FAA4, FAA3 and FAA2 to reduce with Fatty acyl-CoA synthetase activity (Enzyme Commission Number 6.2.1.3), Enhanced acetyl CoA carboxylase (Enzyme Commission Number 6.4.1.2) expression conferred by a recombinant endogenous ACC1 gene wherein said gene is operably linked to a heterologous promoter; Heterologous expression of bacterial Pyruvate Formate Lyase (Enzyme commission Number 2.3.1.54) to improve cytosolic levels of acetyl CoA under anaerobic conditions; Overexpression of Formate dehydrogenase (Enzyme Commission Number 1.17.1.9) to produce NADH/H⁺ and metabolize formic acid to CO₂ and H₂O

Embodiment 4. The recombinant microorganism of embodiment 1 and 2 and 3, wherein the conversion of a carbohydrate source to a hydrocarbon is under anaerobic conditions or in presence of a nitrate, a nitrite, a fumarate, a chlorate, a perchlorate or a hypochlorous acid ion using a specific method of brewing to have alternate cycles of anaerobic respiration and fermentation to enhance co-production of amino-acids, alcohol and lipids.

Embodiment 5. The recombinant microorganism of embodiment 1 and 2 and 3, for production of protein rich “Dried Distillers Grains with Solubles” (DDGS) and/or high protein concentrate enriched with essential amino acids: wherein said essential amino acid is selected from the group consisting of Lysine, Threonine, Methionine, Phenylalanine, Isoleucine, Tryptophan, Valine and Leucine.

Embodiment 6. The recombinant microorganism of embodiment 1 and 2 and 3, wherein said Alcohol derivative is selected from the group consisting of Ethanol, Propanol, Butanol, and any combination thereof.

Embodiment 7. The recombinant microorganism of embodiment 1 and 2 and 3, wherein said Lipid derivative is selected from the group consisting of: A fatty aldehyde; a fatty alcohol; a fatty ester; a fatty acid; an unsaturated fatty acid; a branched-chain fatty acid; a branched methoxy fatty acid; a multi-methyl branched acid; a divinyl-ether fatty acid; a w-phenylalkanoic acid; a fatty amide; a biosurfactant; a dicarboxylic acid; and any combination thereof.

Embodiment 8. The recombinant microorganism of embodiment 5, wherein said hydrocarbon or hydrocarbon derivative comprises a carbon backbone of C₄-C₂₀.

Embodiment 9. The recombinant microorganism of embodiment 1, wherein said lipid or lipid derivative comprises a carbon backbone selected from the group consisting of: Cu; C14; C16; C18; C20; C22; C24; and and any combination thereof.

Embodiment 10. The recombinant microorganism of embodiment 1, wherein said phosphoenolpyruvate carboxykinase is encoded by a polynucleotide from a Thermoanaerobacter species, E. coli, S. cerevisiae or C. thermocellum, T.thermophilus.

Embodiment 11. The recombinant microorganism of embodiment 1, wherein said transcarboxylase is encoded by a polynucleotide from a T. thermophilus, E.coli, C.glutamicum, Thermoanaerobacter species, P. freudenreichii, P. acnes, C. thermocellum, C. bescii, C. cellulolyticum, C. kroppenstedtii, B. fragilis, V. parvula, V. gazogenes, P. the rmopropionicum, Candidatus, Cloacamonas acidaminovorans, G. bemidjiensis or D. propionicus.

Embodiment 12. A process for converting a carbohydrate source to a hydrocarbon comprising contacting the carbohydrate source with a recombinant microorganism according to embodiment 1.

Embodiment 13. The recombinant microorganism of embodiment 12, wherein said acylₙ₊₂-ACP is converted to a fatty acid by a chain termination enzyme.

Embodiment 14. The recombinant microorganism of embodiment 13, wherein said chain termination enzyme is selected from a codon optimized C12 acyl-ACP thioesterase from coconut and Cuphea spp,

Embodiment 15. The recombinant yeast microorganism of embodiment 1, wherein said yeast microorganism is selected from the group consisting of Saccharomyces cerevisiae, Klyveromyces lactis, Kluyveromyces marxianus, Pichia pastoris, Yarrowia lipolytica, Hansenula polymorpha, Phaffia rhodozyma, Candida utilis, Arxula adeninivorans, Pichia stipitis, Debaryomyces hansenii, Debaryomyces polymorphus, Schizosaccharomyces pombe, Candida albicans, and Schwanniomyces occidentalis.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

I/we claim

1. A genetically modified eukaryotic microorganism, wherein the genome of said microorganism is modified relative to a parent non-modified microorganism to:
a. express a heterologous phosphoenolpyruvate carboxykinase (Enzyme commission Number 4.1.1.32);
b. increase expression of aspartate aminotransferase (Enzyme Commission Number 2.6.1.1);
c. increase expression of homoaconitase (Enzyme Commission number 4.2.1.36) activity;
d. increase expression of homocitrate synthase activity (Enzyme commission number: 3.3.1);
e. increase expression of homoisocitrate dehydrogenase (Enzyme commission number: 1.1.1.87);
wherein the genetically modified eukaryotic microorganism produces increased levels of essential amino acids such as lysine, leucine, isoleucine, threonine, methionine, phenylalanine, valine, tryptophan and histidine as compared to said non-genetically modified parent microorganism when cultured under comparable anaerobic fermentation conditions.

2. The genetically modified eukaryotic microorganism of claim 1, wherein the genome of said microorganism is further modified relative to said parent non-modified microorganism to:
a. increase expression of 2-aminoadipate aminotransferase (Enzyme commission number 2.6.1.39);
b. decrease expression of branched-chain amino acid aminotransferase (Enzyme Commission Number 2.6.1.42) and/or decrease expression of threonine aldolase (Enzyme Commission number 4.1.2.48).

3. The genetically modified eukaryotic microorganism of claim 1 or 2, wherein the genome of said microorganism is further modified relative to said parent non-modified microorganism to:
a. express heterologous genes encoding bacterial enzymes:
i. holo-[acyl-carrier-protein] synthase (Enzyme Commission Number 2.7.8.7),
ii. acyl carrier protein,
iii. 3-oxoacyl-[acyl-carrier-protein] synthase 1 (Enzyme Commission Number 2.3.1.41),
iv. malonyl CoA-acyl carrier protein transacylase (Enzyme Commission Number 2.3.1.39),
v. 3-oxoacyl-[acyl-carrier-protein] reductase (Enzyme Commission Number 1.1.1.100),
vi. 3-oxoacyl-[acyl-carrier-protein] synthase 3 (Enzyme Commission Number 2.3.1.41),
vii. Enoyl-acyl carrier protein reductase (Enzyme Commission Number 1.3.1.39), and
viii. 3-hydroxyacyl-[acyl-carrier-protein] dehydratase (Enzyme Commission Number 4.2.1.59);
b. silence expression of native fatty Acid synthase complex (Enzyme Commission Number 2.3.1.86/4.2.1.59);
c. silence or reduce expression of native fatty acyl-CoA synthetase activity (Enzyme Commission Number 6.2.1.3); and
d. increase expression of acetyl CoA carboxylase (Enzyme Commission Number 6.4.1.2); wherein the genetically modified eukaryotic microorganism co-produces increased levels of lipids (which includes but not limited to increased levels of free fatty acids of chain length C12 and analogous triglycerides, fatty alcohols, fatty aldehydes, biosurfactants) as compared to said non-genetically modified parent microorganism when cultured under comparable anaerobic fermentation conditions.

4. The genetically modified eukaryotic microorganism of claim 3, wherein the genome of said microorganism is further modified relative to said parent non-modified microorganism to:
a. express a gene encoding bacterial pyruvate formate lyase (Enzyme commission Number 2.3.1.54)
b. express a gene encoding a heterologous acyl-ACP thioesterase (Enzyme commission number 3.1.2.14); and
c. silence expression of native gene(s) encoding acyl-ACP thioesterase.

5. The genetically modified eukaryotic microorganism of claim 3 or 4, wherein the genome of said microorganism is further modified relative to said parent non-modified microorganism to:
a. increase expression of NADH Kinase (Enzyme commission number 2.7.1.86)
b. express genes encoding bacterial enzymes:
i. citrate synthase (Enzyme commission number: 2.3.3.16) and
ii. isocitrate dehydrogenase (Enzyme commission number: 1.1.1.42).

6. The genetically modified eukaryotic microorganism of any one of claims 1 to 5, wherein the genome of said microorganism is further modified relative to said parent non-modified microorganism to:
c. silence expression of native gene(s) encoding glycerol-3-phosphate dehydrogenase (Enzyme Commission Number 1.1.1.8); and/or
d. silence expression of native gene(s) encoding alcohol dehydrogenase enzyme

7. The genetically modified eukaryotic microorganism of any one of claims 1 to 6, wherein the genome of said microorganism is further modified relative to said parent non-modified microorganism to:
a. increase expression of formate dehydrogenase (Enzyme Commission Number 1.17.1.9)

8. The genetically modified eukaryotic microorganism of any one of claims 1 to 7, wherein the microorganism is a species of a genus selected from the group consisting of Saccharomyces, Kluyveromyces, Pachysolen, Candida, Pichia, Schizosaccharomyces, Hansenula, Kloeckera, Schwanniomyces, Rhodutorula, Trichoderma, Aspergillus and Yarrowia.

9. The genetically modified eukaryotic microorganism of claim 8, wherein the species is selected from the group consisting of Schizzosaccharomyces pombe, Pichia pastoris, Pichia stipitis, Yarrowia lipolytica, Pachysolen tannophilus, Rhodotorula gracilis, Hansenula polymorpha, Phaffia rhodozyma, Candida utilis, Arxula adeninivorans, Debaryomyces hansenii, Debaryomyces polymorphus, Schizosaccharomyces pombe, Trichoderma resit, Aspergillus niger, Aspergillus oryzae and Schwanniomyces occidentalis.

10. Dried Distillers Grains with Solubles and/or a high protein concentrate comprising the genetically modified eukaryotic microorganism of any one of claims 1 to 8.

11. A method for producing essential amino acids and one or more co-product comprising:
a. providing a genetically modified eukaryotic microorganism of any one of claims 1 to 9;
b. culturing said microorganism in a nutrient medium under anaerobic conditions; and
c. recovering one or more fractions of the culture obtained in step (b) enriched in essential amino acids.

12. The method according to claim 11, wherein one of said co-product are lipids comprising:
a. providing a genetically modified eukaryotic microorganism of any one of claims 4 to 8;
b. culturing said microorganism in a nutrient medium under anaerobic conditions; and
c. recovering one or more fractions of the culture obtained in step (b) enriched in essential amino acids and lipids.

13. The method according to claim 11 or 12, wherein said nutrient medium comprises a terminal electron acceptor selected from among a nitrate, a nitrite, a fumarate, a chlorate, a perchlorate or a hypochlorous acid ion.

14. The method according to claim 13, wherein culturing in step (b) is performed under alternate cycles of anaerobic respiration and fermentation, wherein use of a genetically modified eukaryotic microorganism of any one of claims 4 to 8 for co-production of essential amino acids and ethanol.
